SEQUENCE LISTING (93 sequences)

SEQ ID NO: 1 - DNA, 178 bp, Mus
  unsure (107), (173), (175): bases designated as “n” at various positions throughout the sequence may be A, T, C or G
tttaattagcgctctatatacattgcggaacttcccccgactgcagcagtttgactttggcacaacatcaagttccatttcttttggacattggattctgttttganagtatgtatgccccaaagcattttcagtgtcatcaggattagttgggcccattcacagtaattcananatc

SEQ ID NO: 2 - DNA, 148 bp, Mus
tagaatacctggatggcttctcttgtccacccgatctcccgtgttaccaatgtgtatggtctccttctcccgaaagtgtacttaatctttgctttctttgcacaatgtctttggttgcaagtcataagcctgaggcaaataaaattcc

SEQ ID NO: 3 - DNA, 203 bp, Mus
gatctggctagacagttattctgaactatggcttcaagatgaacaagacaagcctaaaaggatggagagaggcaatggagataatgttttggaggaagtatgtcactcaagcatgaactctgtttatttagaaatgagattccatatatgtggtacatgtggaaagaatctaaaaagtcctttaaattttttcattccaaaag

SEQ ID NO: 4 - DNA, 336 bp, Mus
  unsure (3), (4), (6), (7), (60), (64), (70), (73), (74), (79),(83),
  (89), (97), (114), (125), (126), (150), (155), (156),(163),
  (164), (169)..(171), (176), (186), (197), (240), (258),(265),
  (282), (289), (292), (306), (315), (325), (333): bases designated as “n” at various positions throughout the sequence may be A, T, C or G
ctnnannagcactcttcttggccagacctctgtccaaggctcattagaaagctggggttntgtncacgtnacnnacttnatcnaaactnttgctgtnttggcataagttgtgtntctggactgtnntgtattcccctctagacaaaggancaacnnaaaagtnnttgcnnnctttnccagaacatnctcaaagcctntgatggaggagcacaaggaccctgtctgctgagggcccatggntcctctcaggggtttctncccaccnaggcagtgccttcattngctagtngtncagttacttgtagnttatctttnaataaatttnaataaaancta

SEQ ID NO: 5 - DNA, 113 bp, Mus
  unsure (60), (68), (90), (95), (101): bases designated as “n” at various positions throughout the sequence may be A, T, C or G
ctagattgtgtggtttgcctcattgtgctatttgcgcactttccttccctgaagaaatanctgtgaancttctttctgttcagtcctaanattcnaaatanagtgagactatg

SEQ ID NO: 6 - DNA, 164 bp, Mus
  unsure (7), (54), (97), (99), (103), (116), (124), (143), (153): bases designated as “n” at various positions throughout the sequence may be A, T, C or G
ctcaagnacgggccaggtaagggcctttaacacaactaaatcaaggtgtgcttncctccgggttctatgcaagcaaggcatacacactgcactctcncnctcnctaaactggaaangtacagtngcagggctggtttcagacnacgtgatgcntgtttacaaac

SEQ ID NO: 7 - DNA, 141 bp, Mus
tttttattcaatatattaaatatattaatcagaaaagtcacatcctataaatccaggaaaatacacaaatataaatcagaatctgtcaatcaccttcttgagtgacagttatgtacacatggaaggagagcggaagagatc

SEQ ID NO: 8 - DNA, 224 bp, Mus
cgatatacaccatcggtctggggccaacgctaatactacttggtgctgccaattgaattctggtttgctgtgaatctctatcaacaagagtatcatttgtgaatgctttaatttattgagaaagaacaagaagatgatggatacattgatacatttgcgcagccttgcagcctgactcaattctgctgttcatcagttttaatgtcctttctgtgtcatacgtg

SEQ ID NO: 9 - DNA, 210 bp, Mus
  variation (35), (53): bases designated as “n” at various positions throughout the sequence may be A, T, C or G
gatcttttttccttcacttattgctgaaaccaagngcacaattcccattaagngaaggatctctgtgctgtaaactaaacaaattgtgcattttttctggggccattgtttttggtttattttgttattttgttttgtttttgtttttttggtttcattttgttttgggttggtccaattttaaaaggaaatactacaataaaaatgtta

SEQ ID NO: 10 - DNA, 163 bp, Mus
gatctgatttgctagttcttcctggtagagttataaatggaaagattacactatctgattaatagtttcttcatactctgcatataatttgtggctgcagaatattgtaatttgttgcacactatgtaacaaaactgaagatatgtttaataaatattgtact

SEQ ID NO: 11 - DNA, 176 bp, Mus
gcgatgttcttctactcacaactcacgttggtggcctgggcctgaacttgactggagctgacactgtggtgtttgtggagcatgactggaaccctatgcgagatctgcaggccatggaccgggcccatcgtattgggcagaaacgtgtggttaatgtctaccggttgataaccaga

SEQ ID NO: 12 - DNA, 123 bp, Mus
gatctggaagggaatgtccaaagagaagaaggaggagtgggaccgcaaggctgaggatgctaggagggagtatgagaaagccatgaaagagtatgaaggaggaagaggggactcatctaaaag

SEQ ID NO: 13 - DNA, 196 bp, Mus
gatcttcgacacagagaaggagaaatacgagattacagagcagcgaaaggctgaccagaaagctgtggatttgcagattttgccaaagattaaagctgttcctcagctccagggctacctgcgctctcagttttccctgacaaacgggatgtatcctcacaaactggtcttctaaattgttaacctaattaaacag

SEQ ID NO: 14 - DNA, 225 bp, Mus
  Mus (30), (35), (38), (41), (51), (55), (58), (62), (85),(88),
  (95), (97), (103), (165), (174), (206), (212), (213),(223): bases designated as “n” at various positions throughout the sequence may be A, T, C or G
actcaatctcttcaaactctttatactggnctatnatnagnggggatgtgncaanatngacnctggtggtgtatgaaagaaaagntcnatggacntnggcatnccaagattgaattcacctgcttcctacgatgtgtgaaactgctaatagcaaaatatctctanggttatgangagtactgtcgttctgcaaatattcacttcanaactanncaccacgttnaa

SEQ ID NO: 15 - DNA, 244 bp, Mus
  unsure (30), (42), (58)..(60), (80), (97), (106), (164), (176),(232): bases designated as “n” at various positions throughout the sequence may be A, T, C or G
ctagataatcccttactgagtctttcttcncaggtgattcanttgagttgacaattannnctaagaattcaatggactantgaggtgcctcagcagntaatagcanttgctgttcttccagaggaccagagttcagtttctcatcccaagttgggctgctcgtnagtgtcggtaantccagcttcaggggcttgaatttatactgaccatgggcacctgtaccccaacacanacacatacacat

SEQ ID NO: 16 - DNA, 233 bp, Mus
  unsure (18), (35), (45), (58), (73), (80), (92), (102), (104),(130),
  (142), (157), (167), (191), (209), (213), (231): bases designated as “n” at various positions throughout the sequence may be A, T, C or G
ctagaagttaatcctgtnaagcatggtaagaatancattctcaanatcttgagttaanaaagatcttggaggnggctggngagatggctcantggttaagancnctgactgctcttccagaggtcctganttcaattcccancaaccacatggtggntcacaaccanctgtaatgatacctgatgccatcntccgtggtgtatctgaanacanctacagtgacagctacancg

SEQ ID NO: 17 - DNA, 260 bp, Mus
ggattttattctaggcttggccagatacaggttggcatcctaggggaggaagataacaatgtcataggtgaatttgttaggagaggcaagacatgggaaatcattgatttcttcagatttctttaaagcaaattagaagataaatgtctaaaagagatacacttaaaaaatggtgaaactataaccccttaaggagagccagatgtggcaggagccaggtctgaaaatggtagctgaagtaagcagaccagcgtaagatc

SEQ ID NO: 18 - DNA, 154 bp, Mus
cgatgagtcagagaggaagtggacagtgcgttattcattacagcaaaggatttcgttggcatcaaaatctaagtttgttttacaaagattgtttttagtactaagctgccttggcagtttgcatttttgagccaaacaaaaatatattattttc

SEQ ID NO: 19 - DNA, 340 bp, Mus
cgattcaattgtataaatgattataatttctttcatggaagcatgatccttctgattaagaactgtaccccatattttatgctggttgtctgcaagcttgtgcgatgatgttatgttcatgttaatcctatttgtaaaatgaagtgttcctgaccttatgttaaaaagagagaagtaaataacagacattattcagttattttgtcctttatcgaaaaaccagatttcatttttcctttttgtttgtgatctcatttggaaataattggcaagttgaggtactttcttcccatgctttgtacaatataaactgttatgcctttcagtgcgttactgtggg

SEQ ID NO: 20 - DNA, 277 bp, Mus
ctagaggtgggaactggctccactccacacagcagccagttagttagtgacggtcagctgcatgcaggggaatgaaggactcggagagaacgttctgtgctatgtgtgttccatagagattaaaaaggaggcctggagccgagcatggtggtgcacgcctttaatcccagcacttgggaggcagagtcaggtggatttctgagttcattgccagcctggtctacagagtgaattccaggacaggcagggctacacagagaaaccctgtctcaaaaaa

SEQ ID NO: 21 - DNA, 66 bp, Mus
ctagaatttgcagtagcattaattcaagcctacgtattcaccctcctagtaagcctatatctacat

SEQ ID NO: 22 - DNA, 121 bp, Mus
  unsure (28), (78): bases designated as “n” at various positions throughout the sequence may be A, T, C or G
ctagacataagatattgtacataaaganaattttttttgcctttaaatagataaaagtatctatcagataaaaatcangttgtaagttatattgaagacaatttgatacataataaaagat

SEQ ID NO: 23 - DNA, 127 bp, Mus
  unsure (7)..(10), (12), (15), (18), (19), (23), (35), (40)..(42),
  (49), (54), (63), (73), (78): bases designated as “n” at various positions throughout the sequence may be A, T, C or G
ggggagnnnncnagnaannagantcgtacgtaaanagaannntggtgcntttanatagaaaangtactatcanataanaatcaggttgtaagttatattgaagacgntttgatacataataaaagat

SEQ ID NO: 24 - DNA, 105 bp, Mus
ctagactgacaaagactttttgtcaacttgtacaatctgaagcaatgtctggcccacagacagctgagctgtaaacaaatgtcacatggaaataaatactttatc

SEQ ID NO: 25 - DNA, 85 bp, Mus
ctctcttgccacccagatggttaggatgattctgaagatgatgacatccgtaagcctggagaatctgaagaataaactgtaccat

SEQ ID NO: 26 - DNA, 133 bp, Mus
  unsure (119): bases designated as “n” at various positions throughout the sequence may be A, T, C or G
atctctggcaggtcaagtctgggacaatctttgacaatttcctcatcaccagtgatgaggcctatgcagccagttctagcgcagctcacactgagagtgtaagaactacgaacaaaatntctattaaattaag

SEQ ID NO: 27 - DNA, 316 bp, Mus
gatctcggaatggacccaactgctcctgctccaccggcggctcctgcacttgcaccagctcctgcgcctgcaagaactgcaagtgcacctcctgcaagaagagctgctgctcctgctgtcccgtgggctgctccaaatgtgcccagggctgtgtctgcaaaggcgccgcggacaagtgcacgtgctgtgcctgatgtgacgaacagcgctgccaccacgtgtaaatagtatcggaccaacccagcgtcttcctatacagttccaccctgtttactaaacccccgttttctaccgagtacgtgaataataaaagcct

SEQ ID NO: 28 - DNA, 136 bp, Mus
attcagacgaatgagactcctccacattggagacaagagatgcagagagctcagagaatgagggtgtcaagtggtgaaagatggatcaaaggggataagagtgagttaaatgaaataaaagaaaatcaaaggagcc

SEQ ID NO: 29 - DNA, 243 bp, Mus
  unsure (1), (4)..(10), (15), (44), (136), (145), (161), (191),(241): bases designated as “n” at various positions throughout the sequence may be A, T, C or G
ngcnnnnnnnccagnaggaggagaagatgactggccagtatcanaatgggataagatgaggcgcgccctggagtacaccatctacaaccaggagctcaacgagacgcgcgctaagctcgacgagctttctgctaancgagaaacnagtggagagaaatccngacaactaagggatgcccagcaggatgcangagacaaaatggaggatattgagcgccaggttagagaactgaaaacaatnat

SEQ ID NO: 30 - DNA, 359 bp, Mus
  unsure (21), (37), (40), (41), (46), (48), (51), (53), (54),(57),
  (59), (62)..(64), (66), (68), (89), (114), (117), (141),
  (176), (263), (267), (351): bases designated as “n” at various positions throughout the sequence may be A, T, C or G
ctcaaggaaaagacagcaccncgtgcctggcatctgntgnnttagntnatntnnaantntcnnntngncctggcaacggttcctgaacnaattaccactccttcttgccagtcnaanagggtgggaaagtccgagccttangacccagtttcagttctggtttcttccctcctgancaccatcggttgttagttgccttgagttgggaacgtttgcatcgacacctgtaaatgtattcattctttaatttatgtaaggttttntgtnctcaattctttaagaaatgacaaattttggttttctactgttcaatgagaacattaggccccagcaacacgtcattgtgtaaanaaataaaa

SEQ ID NO: 31 - DNA, 139 bp, Mus
cgatggctccatcctggcctcactgtccaccttccagcagatcggctcagcaagcaggagtaggatgagtctggcccctccatcgtgcaccgcaaatgcttctaggcggactgttttacaccctttctttgacaaaacc

SEQ ID NO: 32 - DNA, 354 bp, Mus
  unsure (2), (3), (17), (60), (66), (100), (108), (113), (117),(118),
  (123), (128), (129), (151), (153), (154), (168), (189),(190),
  (253), (258), (267), (270), (283), (289), (299), (313),(319),
  (337), (347), (348): bases designated as “n” at various positions throughout the sequence may be A, T, C or G
cnnatgctacatgctgnaggatgcctaaggctgccccccaccatcccctggctctgctgnccggancaaattgcttccagatgtgactttggaaccttcncacccctnacccnaccnntctcnagaanntcttttatttaaaggaggaaanannacatccaagaaaanggggggaggggggatggaaanncgcatcccctttctagccagctgttcccaaaaggtacccttcctctctgctgctccccaaacncaaancccacttcngancctccacctaaancatcangcaagtcacntacaccctgtttancccccnactctctgcttatacccnggaacaattnntgctcg

SEQ ID NO: 33 - DNA, 412 bp, Mus
  unsure (221), (259), (317), (358): bases designated as “n” at various positions throughout the sequence may be A, T, C or G
cgatggtggggatcttactggggaagaggaaggaccattagcacaccatcatgatgtcagatgacaaaatggaagccaagacaccttgaaggtgactttctaggaaggtcttaagcatgtaatgtccctttatcagagggaaggggacaaactcagggcagccctgtccaggtagaaatatttttgcccccctgtctgatgttgatgaggggtcataccanccagggagaccctctgggaggaagctgccacacacaangactctggaagtatccagatgtgagcccagccagggtcctatggttccaaatctgaanaaaaggtttttcacacactccttgctttctgctaagataanaaaggcgtcactctgccagagtgtgactttttacagattaaataaagctgttat

SEQ ID NO: 34 - DNA, 239 bp, Mus
  unsure (220), (226): bases designated as “n” at various positions throughout the sequence may be A, T, C or G
gatctactccattcccctggaaatcatgcagggcaccgggggtgagctgtttgatcacattgtctcctgcatctccgacttcctggactacatggggatcaaaggccccggatgcctctgggcttcaccttctcgtttccctgcaagcagacgagcctatattgcggaatcttgatcacgtggacaaagggattcaaagccaccgactgtgtgggtcacnatgtanccactttactgag

SEQ ID NO: 35 - DNA, 93 bp, Mus
  unsure (45), (54): bases designated as “n” at various positions throughout the sequence may be A, T, C or G
gatctgagttcgaggccagcctggtctacagagtgagttccaggncagccaggnctacacagagaaaccctgtctcgaaaaaacagaaagaga

SEQ ID NO: 36 - DNA, 130 bp, Mus
  unsure (30): bases designated as “n” at various positions throughout the sequence may be A, T, C or G
ctttcattaaaaagaaaccaggggctgganagatggctcagtggttaagagcaccaactgctcttcccgaaggtcctaagttcaaatcccagcaaccacatggtggctaacaaccactcgtaatgagatc

SEQ ID NO: 37 - DNA, 234 bp, Mus
  unsure (6), (18), (27), (34), (35), (63), (96), (98), (105),(107),
  (109), (112), (122), (123), (127), (131), (134), (142),(166),
  (176), (179)..(182), (190), (197), (224): bases designated as “n” at various positions throughout the sequence may be A, T, C or G
atcgcntggctctcctgnggcctggcntacgacnngaaaaggagtgtccacggctgctgtcgnggccacgattaattaaaactgaagtaccgaggntnccccagngncngantgtggggtcnngccnttcntgntccacaanccaacttggcagacgcttactgtnctgtcaactntcnnnngaataccnccacccncatgctaaaatgatgactgacgttaanccatgctggt

SEQ ID NO: 38 - DNA, 251 bp, Mus
cgatgacaaaggagtcctgaggcagattactctgaatgaccttcctgtcggaagatcagtggacgagacactgcgtttggttcaagccttccagtacactgacaagcatggagaagtctgccctgctggctggaaacctggtagtgaaacaataatcccagatccagctggaaaactgaagtatttcgacaagctaaactgaaaagtacttcagttatgatgtttggaccttctcaataaaggtcattgtg

SEQ ID NO: 39 - DNA, 179 bp, Mus
  unsure (45): bases designated as “n” at various positions throughout the sequence may be A, T, C or G
cgatgctgaataagctcctcaaaaagtggtaaatttaaccttttnaaaaaacaagctttctctgtacagctctggctgttttgttctggaatacattctgtagaattgtctggcctctaacttggagatccaactccctctgcctcttgagtgctgggattaatggcatgtgacactgt

SEQ ID NO: 40 - DNA, 219 bp, Mus
  unsure (103), (183), (187), (210): bases designated as “n” at various positions throughout the sequence may be A, T, C or G
cgatgacctcatgccggcccagaagtgaagcctggccctcgccaccatcaggctgccgcttcctaacttattaaccgggcagtgcccgccatgcatccttgangtttgccgcctggcggctgagcccttagcctcgctgtagagacttctgtcgccctgggtagagtttatttttttgatggntaanctgttgctgacactgaaaataanctagggttt

SEQ ID NO: 41 - DNA, 303 bp, Mus
  unsure (191): bases designated as “n” at various positions throughout the sequence may be A, T, C or G
cgatcaatgaaaagatgacgagtttctttcaaatgggcagttactccctgataacttcatagctgcctgcacagagaagaaaatccctgttgtgtttagactacaagagggttatgatcatagctactacttcattgcaactttcatcgctgaccacatcagacaccatgctaagtacctgaatgcatganaagcctcagccaagagaatctcatcaggaggccggaagggaatcaacaggagtgctgacttcctcgcagaagatcatgctcctgcagctgaatcgcttttctgaataaatat

SEQ ID NO: 42 - DNA, 460 bp, Mus
  unsure (7), (29): bases designated as “n” at various positions throughout the sequence may be A, T, C or G
cgatgtntacttcattgccaccctgtcantcctctggaaggtgtccgtcatcaccttggtcagctgtctccccctctatgtcctcaagtacctgcggagacggttctccccacccagctactcgaagctcacttcctaagctgcagggctgcctcgggcagggcctccggcctccggcgctctcccaggaggaggtcaagttccacacgcacgagccgcctctgctggacggtgcagtcatggctggcacatgaggcttcgctgaggcgacactgggcacctaatggggatggaacattggtggaaccggagggagggacctgagagctgtacctatcagaaccttgggtgctaagctgtgctgagggggaagacgtgggaccggatggcccgtctgaggtttgtggggtcactgtgcaagcttccttatggtttgaacctcttgtcatgtgataaaagt

SEQ ID NO: 43 - DNA, 120 bp, Mus
cgatttacgtatttgactgaaatgaaagttccactaaacggtatttgctcttgtgatatgtggcacattgtgatattttcttagtctgttctgtttcatttaaaaaataaaactgctgat

SEQ ID NO: 44 - DNA, 132 bp, Mus
  unsure (8), (33), (49), (123): bases designated as “n” at various positions throughout the sequence may be A, T, C or G
ccgatgtncgataatagtaaataccttaattanttaaataattcattgnattgtttcagagacgtttggaaattactgtatacatttacaacctaatgacttttgtattttatttttcaaaanaaaagctta

SEQ ID NO: 45 - DNA, 242 bp, Mus
  unsure (2), (5), (7)..(10), (14), (16), (21), (23), (26), (29),(38),
  (44), (45), (57), (59), (63), (64), (69), (70), (92),(98),
  (137), (138), (141)..(143), (145), (148), (154), (186),(188),
  (191), (194), (212), (221), (225), (235), (240): bases designated as “n” at various positions throughout the sequence may be A, T, C or G
cnttngnnnntccntncatcncngcngtntgagtcccncccaannagtccatccaananccanngcatnncagctttatcatgacaacaaantggagnaagaagaagatgagtttcggccactgttgaggcaaatcnntgnnnantcntaatanacacctggtccgctcatccttcaacgttgttntntanaanttacctcccagtagaaangctagcaantttnacctgccacnggttnta

SEQ ID NO: 46 - DNA, 126 bp, Mus
  unsure (25), (27), (33), (38), (44), (72), (78), (84), (92),(96): bases designated as “n” at various positions throughout the sequence may be A, T, C or G
cgatcagatgtcacgcgggacacancnccgccncagtnaatggnaatatatttgcatgttaccccaaattancttctntgcatngaacatangtangtgtctttggggacacgtgtgttctactac

SEQ ID NO: 47 - DNA, 383 bp, Mus
  unsure (19), (34), (61), (185), (213), (217), (276), (277),(317),
  (325), (330), (331), (344), (345), (358), (366), (376): bases designated as “n” at various positions throughout the sequence may be A, T, C or G
cgatttacaaatgaacaancaagattacatatantgaaaatccacgcaggacctattacanagcatggtgaaatagattatgaagcaattgtaaagctttcagatggctttaatggagcatgacctgacaaatgtttgtactgaagcaggtatgtttgcaattcgtgccgatcatgattttgtanttcaggaagacttcatgaaagcagtcangaangtggctgactccaagaagctggagtccaagctggactacaaacctgtgtgattcactannagggtttggtggctgcatgacagacattggtttaatgtanacttaacngttanngaaactaatgtanntattggcaatgancttattanaagtgaatanacatgtg

SEQ ID NO: 48 - DNA, 255 bp, Mus
cgatgtttttaattaagaagaaattcactttctcattacctatgaatctgtgccagggcaggtgatttttgagtatgagaactttgtcctctccacagttgtcacaaaaatggttccttctcattgaactattgtggcatgctaattaagaagtgagtgaccacttgggaggcagaggcaggtggatttctgagtttgaggccagcctggtctacaaagtgagttctaagacagccagggctatacagagaaacc

SEQ ID NO: 49 - DNA, 243 bp, Mus
  unsure (6), (92): bases designated as “n” at various positions throughout the sequence may be A, T, C or G
ccaagnaatatggtctaatcaaaggtcgtctgtctgcttttgattgtctacatcacagcaatccctgggaatttctatccattttaaatgcngccgctttcatctgtttagccagcacacccaatggtttcactaactagcccagttgaccttttggaagtttgagccttgagcaccttcaacaaaattgagcactctgattaggatatccactttgcaaataaaaccaaatgttttgtcaac

SEQ ID NO: 50 - DNA, 358 bp, Mus
  unsure (289): bases designated as “n” at various positions throughout the sequence may be A, T, C or G
cgatgaggggaagatgacctgggccggggaggccatcccttatccaagatcacagggaattctgggaagaggttggcctgtggcatcattgcacgctctgccggccttttccagaaccccaagcagatctgctcctgtgatggcctcactatctgggaggagcgaggccggcccattgccggtcaaggccgaaaggactcagcccaacccccagctcacctctaaacagagcctcatgtcaggttatttggtcctcgtagctgaacatcttcttgcagagggagctgcnggcccttgcttgtacaggcctaagtacagggcagataagtgctgtagcctgaacaaattaaattgttac

SEQ ID NO: 51 - DNA, 355 bp, Mus
  unsure (11), (23), (50), (73), (165): bases designated as “n” at various positions throughout the sequence may be A, T, C or G
cgattagctgnggtctctagganatactcgtcactatatgagctcaggangccagctcttagtagctctgaancaggtgaagaatcctcctctgaggaaacagactgggaggaagaagcagcccattaccagccagctaattggtcaagaaaaaagccaaaagcngctggcgaaagtcagcgtactgttcaacctcccggcagtcggtttcaaggtccgccctatgcggagcccccgccctgcgtagtgcgtcagcaatgcgcagaggggcaatgcgcagagaggtgcgcagaggggcagtgcgcagagaggtgcgcagagaggcagtgcgcagagaggcagtgcgcagactcat

SEQ ID NO: 52 - DNA, 213 bp, Mus
cgatttctaaatcagtctcgcctgtgctaggatgaccggtaatgagcctgtttaaaataagacttaaaagtgtcgtgcgttggccgggcggtaggggcgcatgcctttaatttcataacttggaggtagagacaggcggatctttgtgagttcaaggtcagcctggtgtacagagtgacttccagaacagccagggctgttaaacagagaaac

SEQ ID NO: 53 - DNA, 113 bp, Mus
  unsure (11): bases designated as “n” at various positions throughout the sequence may be A, T, C or G
ttgttttgttnttcagatagggtcttacatatcccatgctggtctcaaactcacattatgcatgcggggaaagccatttactgactgatatacccctggccctaagatagatc

SEQ ID NO: 54 - DNA, 108 bp, Mus
cgatcgtcgttctggtaagaagctggaagatggccccaagttcctgaagtctggccatttaagtttaatagtaaaagactggttaatgataacaatgcatcgtaaaac

SEQ ID NO: 55 - DNA, 257 bp, Mus
  unsure (20), (30), (32), (48), (49), (75), (77), (78), (82),(84),
  (95), (110), (112), (126), (130), (155), (158), (171),(179),
  (183), (192), (195), (201), (206), (227), (240), (245): bases designated as “n” at various positions throughout the sequence may be A, T, C or G
cgatcgtcgttctgagtaanaagctggaananggccccaagttcctgnngtctggcgatgctgccatttaagttnannagananaagactggctnatgataacaatgcancntaaaaccttcaggnaggnaacgaatgttgtggaccattttttntgngtgtggcagtttnaagttatnaagntttcaaaancantacttnttaangggaacaacttgacccatcanctgtcacagaatnttgangaccattaacac

SEQ ID NO: 56 - DNA, 151 bp, Mus
  unsure (1), (31), (34), (50), (85), (107): bases designated as “n” at various positions throughout the sequence may be A, T, C or G
nctacgatcatctagatctactagacctacnacnagaccatgggccaaanatggtcgacctgcaaacttgcaaggtttattttanatacacattatggcgttttatnttttgtaattctaagttgtaattcagcttttaacaaatcttttt

SEQ ID NO: 57 - DNA, 152 bp, Mus
  unsure (6), (8), (12), (32), (40), (43), (59), (60), (67),(71), (80),
  (82), (90), (92), (101), (109), (122), (126), (131),(138), (149): bases designated as “n” at various positions throughout the sequence may be A, T, C or G
ccaagnanatcnagactactagacctactacnagaccatnggncaaacatggtcgaccnncaaacgnatangtatatttnanatacacananatagcgttntatgtctngtaattctaagtngtanatcanctattancaaaatctttnttt

SEQ ID NO: 58 - DNA, 188 bp, Mus
  unsure (41), (72), (84), (95), (106), (165), (177): bases designated as “n” at various positions throughout the sequence may be A, T, C or G
cgatggaagttctgctgagcccttctgacgtaaccctggcnatggctaacactgtccttcctgcaatgttcntggtggacacancttctctgganataccctgaangtggcacgccctgttccagcccacctggtgtgcactttttgccctctttacctcattantaaatgttttcntgctcctaatg

SEQ ID NO: 59 - DNA, 136 bp, Mus
  unsure (3), (6), (12), (127): bases designated as “n” at various positions throughout the sequence may be A, T, C or G
ctnagnaagganctgtacttcgtattgcaaggcagtctcttgtgtcttcttagagtgtcttccccatgcacagcctcagtttggagcactagtttataatgtttattacaatttttaataaattgantaggtagta

SEQ ID NO: 60 - DNA, 365 bp, Mus
  unsure (3), (6), (84), (192), (208), (228), (265), (274),(277),
  (315), (323), (343): bases designated as “n” at various positions throughout the sequence may be A, T, C or G
tcntcnttctggtaagaactggaatatggccccaagttcctgaagtctggcgatgctgccattgttgatatggtccctggcaancccatgtgtgttgagagcttctctgactaccctccacttggtcgctttgctgttcgtgacatgaggcagacagttgctgtgggtgtcatcaaagctgtggacaaaaangctgctggagctggcnaagtcaccaagtctgcccanaaagctcagaaggctaaatgaatattacccctaacanctgccaccncantcttaatcagtggtggaagaacggtctcagaactgttngtctcaantggccatttaagtttaatantaaaagactggttaatgataac

SEQ ID NO: 61 - DNA, 357 bp, Mus
  unsure (6), (21), (22), (24), (45), (47), (48), (58), (61),(66), (98),
  (112), (113), (116), (125), (177), (182), (193), (195),(215),
  (223), (252), (267), (284), (304), (323), (334), (338),(346): bases designated as “n” at various positions throughout the sequence may be A, T, C or G
cgatcntcgttctggtaaganncnggaacatggccccaagttccnganntctggcgangcngccantgttgatatggtccctggcaagcccatgtgtnttgagagcttcacnnacnaccctccanttggtcgctttgctgttcgtgacatgaggcagacagttgctgtgggtgtcancaaanctgtggacaananggctgctggagctggcaagntcaccaantctgcccagaaagctcagaatgctaaatnaatattacccctaanacctgccaccccagtcntaatcagtggtggaataacngtctcagaactgtttgtcncaattggccanttangtttaatnatacaagactg

SEQ ID NO: 62 - DNA, 305 bp, Mus
  unsure (2)..(12), (14), (16), (159): bases designated as “n” at various positions throughout the sequence may be A, T, C or G
gnnnnnnnnnnncnangaaaaagaggtgaaaaatgcttggctctagctgatgacagaaagctgaaatccatcgccttcccatccattggcagcggcaggaacgggttcccggaagcagacagcggcccagctcattctgaagtgccatctccagctacnttgtctccacgatgtcctcctccatcaaaactgtgtacttcatgctttttgacagtgagagcataggtatctatgtgcaggaaatggccaagctggacgccaactaggccagtgatccctagagccagcacatgcggtgtccccca

SEQ ID NO: 63 - DNA, 327 bp, Mus
  unsure (3), (5), (21), (149), (166), (203), (233), (283),(291), (297): bases designated as “n” at various positions throughout the sequence may be A, T, C or G
ctnangaaagctgctggggcnccctgacatcactcatcactcactatgctaccaattctatttatttcggaattacaagatatcgggaatctctctgcaggctggactggcaggctgtggggtgggcgggacacggctcttaacatttncagagggaaacgcgcanatgtccaaaagtctaaataaatgcattcagaggtttntggggtccatggccaagtggagttcccccncagggggaggtggggtaagtgcctccaggaaggcaggcagcctgccttanacttgcancccggntgtgggaatgaatcattggagtaataaact

SEQ ID NO: 64 - DNA, 271 bp, Mus
cgatgccaatggcatcctcaatgtttctgctgtagataagagcacaggaaaggagaaagtctgcaaccctatcattaccaagctgtaccagagtgcaggtggcatgcctgggggaatgcctggtggcttcccaggtggaggagctcccccatctggtggtgcttcttcaggccccaccattgaagaggtggattaagtcagtccaagaagaaggtgtagctttgttccacagggacccaaaacaagtaacatggaataataaaactattta

SEQ ID NO: 65 - DNA, 310 bp, Mus
cgatgaagatgaggtcactgcagaggagcccagtgctgctgttcctgatgagatcccccctctggaaggcgatgaggatgcctcgcgcatggaagaggtggattaaagcctcctggaagaagccctgccctctgtatagtatccccgtggctcccccagcagccctgacccacctggatctctgctcatgtctacaagaatcttctatcctgtcctgtgccttaaggcaggaagatcccctcccacagaatagcagggttgggtgttatgtattgtggtttttttgtttgttttattttgttctaaaatt

SEQ ID NO: 66 - DNA, 579 bp, Mus
  unsure (491), (498), (505), (511), (512), (521), (523), (542),(555): bases designated as “n” at various positions throughout the sequence may be A, T, C or G
cgatgccaatggcatcctcaatgtttctgctgtagataagagcacaggaaaggagaacaagatcaccatcaccaatgacaagggccgcttgagtaaggaagatattgagcgcatggtccaagaagctgagaagtacaaggctgaggatgagaagcagagagataaggtttcctccaagaactcactggagtcctatgccttcaacatgaaagcaactgtggaagatgagaaacttcaaggcaagatcaatgatgaggacaaacagaagattcttgacaagtgcaatgaaatcatcagctggctggataagaaccagactgcagagaaggaagaatttgagcatcagcagaaagaactggagaaagtctgcaaccctatcattaccaagctgtaccagagtgcaggtggcatgcctgggggaatgcctggtggcttcccaggtggaggagctcccccatctggtggtgcttcttcaggccccaccattgaanaggtggnttaagtnatccannaagaaaggntncctttttttccaaaggganccaaaaaagtaanatggataataaaacctatttaatt

SEQ ID NO: 67 - DNA, 186 bp, Mus
  unsure (13), (15), (21), (23), (29), (32), (44): bases designated as “n” at various positions throughout the sequence may be A, T, C or G
cgatgccaatagnancccaantntctgcngtngataagacacangaaaagagaacaagatcaccatcaccaatgacaagggccgcttgagtaaggaagatattgagcgcatggtccaagatcaatgatgaggacaaacagaagattcttgacaagtgcaatgaaatcatcagctggctggataaga

SEQ ID NO: 68 - DNA, 321 bp, Mus
cgattagcggaggtctctaggagatactcgtcactagatgagctcaggaagccagctcttagtagctctgaagcaagtgaagaatcctcctctgaggaaacagactgggaggaagaagcagcccattaccagccagctaattggtcaagaaaaaagccaaaagcggctggcgaaagtcagcgtactgttcaacctcccggcagtcggtttcaaggtccgccctatgcggagcccccgccctgcgtagtgcgtcagcaatgcgcagaggggcaatgcgcagagaggcagtgcgcagagaggcagtgcgcagactcattcatt

SEQ ID NO: 69 - DNA, 205 bp, Mus
  unsure (202): bases designated as “n” at various positions throughout the sequence may be A, T, C or G
tctctgtataaccctggatgtcctggaactcactttgtagaccaggttggcctcgaactcagaaatccgcctgcctctgccaagcgctgggattaaaggtgtgcgccaccacacccggcaggtaatttttttctttttaaagatttattatgtatacaggttctgcctacatgtgtacctgccggccagaagagggcatcanatc

SEQ ID NO: 70 - DNA, 495 bp, Mus
gatctttgtaggcacaaaatgaatcccgcacctggtgacccatgatgctcgtactattcggtaccctgatcccctcatcaaggtgaacgacaccattcagattgatttggagacaggcaaaataactgacttcatcaagtttgacactgggaacctgtgtatggtgactggaggtgctaacttgggaagaattggtgtaatcaccaacagagagagacatcccggctcttttgatgtggttcatgtgaaagatgccaatggcaacagctttgccactcggctgtccaacatttttgttattggcaagggtaacaaaccatggatctctcttcccagaggaaaaggaatccgcctcaccattgctgaagagagagacaagaggcttgcggccaaacagagcagtgggttgaaatggtctcctaggagacatgcctggaaagttgttttgtacaacctttcctaggcaacatacattgctagaattaaacagccatg

SEQ ID NO: 71 - DNA, 136 bp, Mus
cgatcgagagggcaaaccacggaaggtggttggttgcagttgcgtagtggttaaggactatggcaaagaatctcaggccaaggatgtcatcgaggaaatacttcaagtgcaagaaataaataaattttggctgatt

SEQ ID NO: 72 - DNA, 140 bp, Mus
attccagatgaggaccacaagcgactcattgatttacatagtccttctgagattgttaagcagattacttccatcagtattgagccgggagttgaggttgaagtcaccattgcagatgcctaagacaactgaataaatcg

SEQ ID NO: 73 - DNA, 216 bp, Mus
gatctatacagtcgggaaacgcttcaaggaagcaaataacttcctgtggcccttcaagttatcttccccacgaggtgggatgaagaaaaagacaactcactttgtagaaggtggagatgctggcaacagggaagaccagataaacaggcttattagacggatgaactaaggtgtcacccattgtatttttgtaatctggtcagttaataaacagtc

SEQ ID NO: 74 - DNA, 151 bp, Mus
cgatgtggccaaagtcaataccctgataaggcccgacggagagaagaaggcgtatgttcgcttggctcctgattatgatgccctagatgttgccaacaagattgggatcatctaaactgagtccagatggctaattctaaatatatacttt

SEQ ID NO: 75 - DNA, 90 bp, Mus
  unsure (52), (54), (73): bases designated as “n” at various positions throughout the sequence may be A, T, C or G
gatctggaaccatagatgcgagcatcagcaacagaatacaagaaatggaagngngaatctcaggtgcagaagnttccatagagaacatcg

SEQ ID NO: 76 - DNA, 257 bp, Mus
  variation (21), (99), (101), (141), (151), (157), (162), (186),(231),
  (240): bases designated as “n” at various positions throughout the sequence may be A, T, C or G
gcgatgcaaaatccttaatanaattcttgctaaccgaatccaagaacacattaaagcaatcatccatcctgaccaagtaggttttattccagggatgcngngatggtttaatatatgaaaatccatcaatgtaatccattntataaacaanctcaangacanaaaccacatgatcatctcgttagntgcagaaaaagcatttgacaagatccaacacacattcgtgataanagttttggnaagatcaggaattcaag

SEQ ID NO: 77 - DNA, 200 bp, Mus
  unsure (5), (6), (74), (94), (102), (116), (137), (169), (179),(191): bases designated as “n” at various positions throughout the sequence may be A, T, C or G
cgatnnacccgctctacctcaccatctcttgctaattcagcctatataccgccatcttcagcaaaccctaaatnaggtattaaagtaagcatcnagaatcanccatactcaacgtnacgtcaaggtgtacccaatgnaatgggaagaaatgggctacattttcttatanaagaacattnctataccctttntgaaactaa

SEQ ID NO: 78 - DNA, 56 bp, Artificial Sequence
  variation (56): nucleotide degeneracy; v = a or c or g
  Description of Artificial Sequence  oligo used in gene expression
acgtaatacgactcactatagggcgaattgggtcgacttttttttttttttttttv

SEQ ID NO: 79 - DNA, 21 bp, Artificial Sequence
  Description of Artificial Sequence  oligo used in gene expression
cttacagcggccgcttggacg

SEQ ID NO: 80 - DNA, 15 bp, Artificial Sequence
  Description of Artificial Sequence  oligo used in gene expression
agcggccgctgtaag

SEQ ID NO: 81 - DNA, 30 bp, Artificial Sequence
  Description of Artificial Sequence  oligo used in gene expression
gcggaattccgtccaagcggccgctgtaag

SEQ ID NO: 82 - DNA, 21 bp, Artificial Sequence
  Description of Artificial Sequence  adapter oligo
cttacagcggccgcttggacg

SEQ ID NO: 83 - DNA, 15 bp, Artificial Sequence
  Description of Artificial Sequence  adapter oligo
gaatgtcgccggcga

SEQ ID NO: 84 - DNA, 25 bp, Artificial Sequence
  Description of Artificial Sequence  adapter oligo
tagcgtccggcgcagcgacggccag

SEQ ID NO: 85 - DNA, 29 bp, Artificial Sequence
  Description of Artificial Sequence  adapter oligo
gatcctggccgtcggctgtctgtcggcgc

SEQ ID NO: 86 - DNA, 30 bp, Artificial Sequence; identical to SEQ ID NO: 81
  Description of Artificial Sequence  PCR primer

SEQ ID NO: 87 - DNA, 40 bp, Artificial Sequence
  Description of Artificial Sequence  PCR primer
ctctcaaggatcttaccgctttttttttttttttttttat

SEQ ID NO: 88 - DNA, 40 bp, Artificial Sequence
  Description of Artificial Sequence  PCR primer
taataccgcgccacatagcattttttttttttttttttcg

SEQ ID NO: 89 - DNA, 40 bp, Artificial Sequence
  Description of Artificial Sequence  PCR primer
cagggtagacgacgctacgcttttttttttttttttttga

SEQ ID NO: 90 - DNA, 19 bp, Artificial Sequence
  Description of Artificial Sequence  PCR primer
tagcgtccggcgcagcgac

SEQ ID NO: 91 - DNA, 19 bp, Artificial Sequence
  Description of Artificial Sequence  PCR primer
ctctcaaggatctaccgct

SEQ ID NO: 92 - DNA, 20 bp, Artificial Sequence
  Description of Artificial Sequence  PCR primer
cagggtagacgacgctacgc

SEQ ID NO: 93 - DNA, 20 bp, Artificial Sequence
  Description of Artificial Sequence  PCR primer
taataccgcgccacatagca

(19) United States
(12) Patent Application Publication
(10) Pub. No.: US 2007/0154930 A1
(43) Pub. Date: Jul. 5, 2007

(54) Process to study changes in gene expression in stem cells

(76) Inventors: Meng Liu, New Haven, CT (US); Sherman M. Weissman, New Haven, CT (US); Namadev Baskaran, Acton, MA (US)

Correspondence Address: MORGAN LEWIS & BOCKIUS LLP, 1111 PENNSYLVANIA AVENUE NW, WASHINGTON, DC, 20004, US

(21) Appl. No.: 11/633,033

(22) Filed: Dec. 4, 2006

Related U.S. Application Data
(63) Continuation of application No. 09/506,731, filed on Feb. 18, 2000, now abandoned.
Application No. 09/506,731 is a continuation of application No. PCT/US98/17283, filed on Aug. 21, 1998.
(60) Provisional application No. 60/056,861, filed on Aug. 22, 1997.

(51) Int. Cl. C12Q 1/68

(52) U.S. Cl. 435/6

(57) ABSTRACT

The present invention includes a method to identify stem cell genes that are differentially expressed in stem cells at various stages of differentiation when compared to undifferentiated stem cells by preparing a gene expression profile of a stem cell population and comparing the profile to a profile prepared from stem cells at different stages of differentiation, thereby identifying cDNA species, and therefore genes, which are expressed. The present invention also includes methods to identify a therapeutic agent that modulates the expression of at least one stem cell gene associated with the differentiation, proliferation and/or survival of stem cells.

### TECHNICAL FIELD

This invention relates to compositions and methods useful to identify agents that modulate the expression of at least one gene associated with the differentiation, proliferation, dedication and/or survival of stem cells.

### BACKGROUND OF THE INVENTION

The identification of genes associated with development and differentiation of cells is an important step for advancing our understanding of hematopoiesis, the differentiation of hematopoietic stem cells into erythrocytes, monocytes, platelets and polymorphonuclear white blood cells or granulocytes. The identification of genes associated with hematopoiesis is also an important step for advancing the development of therapeutic agents which modulate, promote or interfere with the differentiation of stem cells.

Hematopoietic stem cells derive from bone marrow stem cells. The bone marrow stem cells ultimately differentiate into the hematopoietic stem cells, which are responsible for the lymphoid, myeloid and erythroid lineages, and stromal stem cells, which differentiate into fibroblasts, osteoblasts, smooth muscle cells, stromal cells and adipocytes (STEWART SELL, IMMUNOLOGY, IMMUNOPATHOLOGY & IMMUNITY, 5th ed. 39-42 Stamford, Conn., 1996). The lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as others cells, monitors for the presence of foreign bodies in the blood stream, provides protection against neoplastic cells, scavenges foreign materials in the blood stream, produces platelets and the like. The erythroid lineage provides the red blood cells which act as oxygen carriers.

Hematopoietic stem cells differentiate as a result from their interaction with growth factors such as interleukins (ILs), lymphokines, colony-stimulating factors (CSFs), erythropoietin (epo), and stem cell factor (SCF). Each of these growth factors have multiple actions that are not necessarily limited to the hematopoietic system (ROBERT A. MEYERS, ED., MOLECULAR BIOLOGY AND BIOTECHNOLOGY: A COMPREHENSIVE DESK REFERENCE, 392-6, New York, 1995). Proliferation, differentiation and survival of immature hematopoietic progenitor cells are sustained by hematopoietic growth factors (hemopoietins). These growth factors also influence the survival and function of mature blood cells. The kinetics of hematopoiesis vary depending on cell type, and their life span may-be as little as 6-12 hours to as much as months or years. As a result, the daily renewal of certain lymphocyte progenitors may be substantially lower than that of leukocytic progenitors. The most primitive cells, pluripotent stem cells. (PSCs), have high self-renewal capacity (Nathan, 818-821; Saito, Recent trends in research on differentiation of hematopoietic cells and lymphokines, Hum. Cell. 5(1): 54 (1992)).

Growth factors are responsible for differentiating the hematopoietic stem cell into either the hemocytoblast, which is the progenitor cell of erythrocytes, neutrophils, eosinophils, basophils, monocytes and platelets, and lymphoid stem cells, which are progenitors to T cells and B cells. SELL, 41. These circulating blood cells are products of terminal differentiation of recognizable precursors (e.g., erythroblasts, mono-myeloblasts and megakaryoblasts, to name but a few). The terminal differentiation of these recognizable precursors may occur exclusively in the marrow cavities of the axial skeleton, with some extension into the proximal femora and humeri (David G. Nathan, Hematologic Diseases, IN CECIL TEXTBOOK OF MEDICINE 20th ed., 817, Philadelphia, 1996). White blood cell (WBC) nomenclature may be divided into two major populations on the basis of the form of their nuclei: single nuclei (mononuclear or “round cells”) or segmented nuclei (polymorphonuclear).

In human medicine, the ability to initiate and regulate hematopoiesis is of great importance (McCune et al., The SCID-hu mouse: murine model for the analysis of human hematolymphoid differentiation and function, Science 241: 1632(1988)). A variety of diseases and immune disorders, including malignancies, appear to be related to disruptions within the lympho-hematopoietic system. Many of these disorders could be alleviated and/or cured by repopulating the hematopoietic system with progenitor cells, which when triggered to differentiate would overcome the patient's deficiency. In humans, a current replacement therapy is bone marrow transplantation. This type of therapy, however, is both painful (for donor and recipient) because of involvement of invasive procedures and can offer severe complications to the recipient, particularly when the graft is allogeneic and Graft Versus Host Disease (GVHD) results. Therefore, the risk of GVHD restricts the use of bone marrow transplantation to patients with otherwise fatal diseases. A potentially more exciting alternative therapy for hematopoietic disorders is the treatment of patients with reagents that regulate the proliferation and differentiation of stem cells (Lawman et al., U.S. Pat. No. 5,650,299 (1997)).

There is also a strong interest in the development of procedures to produce large numbers of the human hematopoietic stem cell. This will allow for identification of growth factors associated with its self regeneration. Additionally, there may be as yet undiscovered growth factors associated (1) with the early steps of dedication of the stem cell to a particular lineage; (2) the prevention of such dedication; and (3) the negative control of stem cell proliferation. Availability of large numbers of stem cells would be extremely useful in bone marrow transplantation, as well as transplantation of other organs in association with the transplantation of bone marrow.

An in vitro system that permits determination of what agents induce differentiation or proliferation of progenitor cells within a hematopoietic cell population would have many applications. For example, controlled production of red blood cells would permit the in vitro production of red blood cell units for clinical replacement (transfusion) therapy. As is well known, transfused red cells are used in the treatment of anemia following elective surgery, in cases of traumatic blood loss, and in the supportive care of, e.g., cancer patients. Similarly, controlled production of platelets would permit the in vitro production of platelets for platelet transfusion therapy, which may be used in cancer patients with thrombocytopenia caused by chemotherapy. For both red cells and platelets, current volunteer donor pools are accompanied by the risk of infectious contamination, and availability of an adequate supply can be limited. Determination of such compounds would lend itself to developing methods of controlled in vitro production of specified lineage of mature blood cells to circumvent these problems (Palsson et al., U.S. Pat. No. 5,635,386 (1997)).

Alternatively, agents could be isolated that selectively deplete a particular lineage of cells from within a hematopoietic cell population and can similarly confer important advantages. For example, production of stem cells and myeloid cells while selectively depleting T-cells from a bone marrow cell population could be very important for the management of patients with human immunodeficiency virus (HIV) infection. Since the major reservoir of HIV is the pool of mature T-cells, selective eradication of the mature T-cells from a hematopoietic cell mass collected from a patient has considerable potential therapeutic benefit. If one could selectively remove all the mature T-cells from within an HIV infected bone marrow cell population while maintaining viable stem cells, the T-cell depleted bone marrow sample could then be used to “rescue” the patient following hematolymphoid ablation and autologous bone marrow transplantation. Although there are reports of the isolation of progenitor cells (see, e.g., Tsukamoto et al., (1991) as representative) such techniques are distinct from the selective removal of T-cells from a hematopoietic tissue culture (Palsson et al., U.S. Pat. No. 5,635,386 (1997)).

### SUMMARY OF THE INVENTION

While the differentiation of stem cells has been the subject of intense study, little is known about the global transcriptional response of stem cells during cell hematopoiesis. The present inventors have devised an approach to systematically assess the transcriptional regulation of stem cells during hematopoiesis as well as methods for the identification of agents that modulate the expression of at least one gene associated with hematopoiesis.

The present invention includes a method to identify stem cell genes that are differentially expressed in stem cells at various stages of differentiation when compared to undifferentiated stem cells by preparing a gene expression profile of a stem cell population and comparing the profile to a profile prepared from stem cells at different stages of differentiation, thereby identifying cDNA species, and therefore genes, which are expressed.

The present invention further includes a method to identify an agent that modulates the expression of at least one stem cell gene associated with the differentiation process of a stem cell population, comprising the steps of preparing a first gene expression profile of an undifferentiated stem cell population, preparing a second gene expression profile of a stem cell population at a defined stage of differentiation, treating said undifferentiated stem cell population with the agent, preparing a third gene expression profile of the treated stem cell population, and comparing the first, second and third gene expression profiles. Comparison of the three gene expression profiles for RNA species as represented by cDNA fragments that are differentially expressed upon addition of the agent to the undifferentiated stem cell population identifies agents that modulate the expression of at least one gene in undifferentiated stem cells that is associated with stem cell differentiation.

Another aspect of the invention is a composition comprising a grouping of nucleic acids or nucleic acid fragments affixed to a solid support. The nucleic acids affixed to the solid support correspond to one or more genes whose expression levels are modulated during stem cell differentiation.

### BRIEF DESCRIPTION OF THE DRAWINGS

FIG. 1 FIG. 1 is an autoradiogram of the gene expression profiles generated from cDNAs made with RNA isolated from Lin⁺, LRH, LRH48 and LRBRH cells. All possible 12 anchoring oligo d(T)n1, n2 were used to generate a complete expression profile for the enzyme ClaI.

### MODES OF CARRYING OUT THE INVENTION

### General Description

The differentiation of stem cells during the process of hematopoiesis is a subject of primary importance in view of the need to find ways to modulate the stem cell differentiation process. One means of characterizing the process of hematopoiesis is to measure the ability of stem cells to synthesize specific RNA during stem cell differentiation.

The following discussion presents a general description of the invention as well definitions for certain terms used herein.

Definitions

The term “stem cells” as used herein, refers to both hematopoietic stem cells and bone marrow stem cells, and includes totipotent cells which serve as progenitors of neoplastic transformation. The term “hematopoietic stem cells” refers to stem cells which differentiate into erythrocytes, monocytes, granulocytes, and platelets. The putative human hematopoietic stem cell may express the cell surface antigen CD34.

The term “hematopoiesis” as used herein, refers to the process by which stem cells differentiate into blood cells, including erythrocytes, monocytes, granulocytes, and platelets.

The term “blood cell”, as used herein, refers to all blood cell types derived from the process of hematopoiesis (see STEWART SELL, IMMUNOLOGY, IMMUNOPATHOLOGY & IMMUNITY, 5th ed. 39-42, Stamford, Conn., 1996)

The term “solid support”, as used herein, refers to any support to which nucleic acids can be bound or immobilized, including nitrocellulose, nylon, glass, other solid supports which are positively charged and nanochannel glass arrays disclosed by Beattie (WO 95/1175).

The term “gene expression profile”, also referred to as a “differential expression profile” or “expression profile” refers to any representation of the expression level of at least one mRNA species in a cell sample or population. For instance, a gene expression profile can refer to an autoradiograph of labeled cDNA fragments produced from total cellular mRNA separated on the basis of size by known procedures. Such procedures include slab gel electrophoresis, capillary gene electrophoresis, high performance liquid chromatography, and the like. Digitized representations of scanned electrophoresis gels are also included as are two and three dimensional representations of the digitized data.

While a gene expression profile encompasses a representation of the expression level of at least one mRNA species, in practice, the typical gene expression profile represents the expression level of multiple mRNA species. For instance, a gene expression profile useful in the methods and compositions disclosed herein represents the expression levels of at least about 5, 10, 20, 50, 100, 150, 200, 300, 500, 1000 or more preferably, substantially all of the detectable mRNA species in a cell sample or population. Particularly preferred are gene expression profiles or arrays affixed to a solid support that contain a sufficient representative number of mRNA species whose expression levels are modulated under the relevant infection, disease, screening, treatment or other experimental conditions. In some instances a sufficient representative number of such mRNA species will be about 1, 2, 5, 10, 15, 20, 25, 30, 40, 50, 50-75 or 100.

Gene expression profiles can be produced by any means known in the art, including, but not limited to the methods disclosed by: Prashar et al. (1996) Proc. Natl. Acad. Sci. USA 93:659-663; Liang et al. (1992) Science 257:967-971; Ivanova et al. (1995) Nucleic Acids Res. 23:2954-2958; Guilfoyl et al. (1997) Nucleic Acids Res. 25(9):1854-1858; Chee et al. (1996) Science 274:610-614; Velculescu et al. (1995) Science 270:484-487; Fischer et al. (1995) Proc. Natl. Acad. Sci. USA 92(12):5331-5335; and Kato (1995) Nucleic Acids Res. 23(18):3685-3690.

As an example, gene expression profiles are made to identify one or more genes whose expression levels are modulated during the process of stem cell differentiation. The assaying of the modulation of gene expression via the production of a gene expression profile generally involves the production of cDNA from polyA⁺ RNA (mRNA) isolated from stem cells as described below.

Stem cells are harvested or isolated by any technique known in the art. One of the most versatile ways to separate hematopoietic cells is by use of flow cytometry, where the particles, i.e., cells, can be detected by fluorescence or light scattering. The source of the cells may be any source which is convenient. Thus, various tissues, organs, fluids, or the like may be the source of the cellular mixtures. Of particular interest are bone marrow and peripheral blood, although other lymphoid tissues are also of interest, such as spleen, thymus, and lymph node (see Sasaki et al., U.S. Pat. No. 5,466,572 and Fei et al., U.S. Pat. No. 5,635,387).

Cells of interest will usually be detected and separated by virtue of surface membrane proteins which are characteristic of the cells. For example, CD34 is a marker for immature hematopoietic cells. Markers for dedicated cells may include CD 10, CD 19, CD20, and sIg for B cells, CD 15 for granulocytes, CD 16 and CD33 for myeloid cells, CD 14 for monocytes, CD41 for megakaryocytes, CD38 for lineage dedicated cells, CD3, CD4, CD7, CD8 and T cell receptor (TCR) for T cells, Thy-1 for progenitor cells, glycophorin for erythroid progenitors and CD71 for activated T cells. In isolating early progenitors, one may divide a CD34 positive enriched fraction into lineage (Lin) negative, e.g. CD2−, CD 14−, CD15−, CD16−, CD10−, CD19−, CD33− and glycophorin A−, fractions by negatively selecting for markers expressed on lineage committed cells, Thy-1 positive fractions, or into CD38 negative fractions to provide a composition substantially enriched for early progenitor cells. Other markers of interest include V alpha and V beta chains of the T-cell receptor (Sasaki et al., U.S. Pat. No. 5,466,572(1995)).

After isolation of the appropriate stem cells, total cellular mRNA is isolated from the cell sample. mRNAs are isolated from cells by any one of a variety of techniques. Numerous techniques are well known (see e.., Sambrook et al., Molecular Cloning: A Laboratory Approach, Cold Spring harbor Press, NY, 1987; Ausbel et., Current Protocols in Molecular Biology, Greene Publishing Co. NY, 1995). In general, these techniques first lyse the cells and then enrich for or purify RNA. In one such protocol, cells are lysed in a Tris-buffered solution containing SDS. The lysate is extracted with phenol/chloroform, and nucleic acids precipitated. The mRNAs may be purified from crude preparations of nucleic acids or from total RNA by chromatography, such as binding and elution from oligo(dT)-cellulose or poly(U)-Sepharose®. However, purification of poly(A)-containing RNA is not a requirement. As stated above, other protocols and methods for isolation of RNAs may be substituted.

The mRNAs are reverse transcribed using an RNA-directed DNA polymerase, such as reverse transcriptase isolated from AMV, MoMuLV or recombinantly produced. Many commercial sources of enzyme are available (e.g. Pharmacia, New England Biolabs, Stratagene Cloning Systems). Suitable buffers., cofactors, and conditions are well known and supplied by manufacturers (see also, Sambrook et al. (1989) Molecular Cloning: a laboratory manual, 2nd Ed., Cold Spring Harbor Laboratory; and Ausbel et al., (1987) Current Protocols in Molecular Biology, Greene Publishing and Wiley-Interscience, N.Y.).

Various oligonucleotides are used in the production of cDNA. In particular, the methods utilize oligonucleotide primers for cDNA synthesis, adapters, and primers for amplification. Oligonucleotides are generally synthesized so single strands by standard chemistry techniques, including automated synthesis. Oligonucleotides are subsequently de-protected and may be purified by precipitation with ethanol, chromatographed using a sized or reversed-phase column, denaturing polyacrylamide gel electrophoresis, high-pressure liquid chromatography (HPLC), or other suitable method. In addition, within certain preferred embodiments, a functional group; such as biotin, is incorporated preferably at the 5′ or 3′ terminal nucleotide. A biotinylated oligonucleotide may be synthesized using pre-coupled nucleotides, or alternatively, biotin may be conjugated to the oligonucleotide using standard chemical reactions. Other functional groups, such as florescent dyes, radioactive molecules, digoxigenin, and the like, may also be incorporated.

Partially-double stranded adaptors are formed from single stranded oligonucleotides by annealing complementary single-stranded oligonucleotides that are chemically synthesized or by enzymatic synthesis. Following synthesis of each strand, the two oligonucleotide strands are mixed together in a buffered salt solution (e.g., 1 M NaCl, 100 mM Tris-HCl pH.8.0, 10 mM EDTA) or in a buffered solution containing Mg⁺² (e.g., 10 mM MgCl₂) and annealed by heating to high temperature and slow cooling to room temperature.

The oligonucleotide primer that primes first strand DNA synthesis may comprise a 5′ sequence incapable of hybridizing to a polyA tail of the mRNAs, and a 3′ sequence that hybridizes to a portion of the polyA tail of the mRNAs and at least one non-polyA nucleotide immediately upstream of the polyA tail. The 5′ sequence is preferably a sufficient length that can serve as a primer for amplification. The 5′ sequence also preferably has an average G+C content and does not contain large palindromic sequence; some palindromes, such as a recognition sequence for a restriction enzyme, may be acceptable. Examples of suitable 5′ sequences are CTCTCAAGGATCTACCGCT (SEQ ID No.______), CAGGGTAGACGACGCTACGC (SEQ ID No.______ ), and TAATACCGCGCCACATAGCA (SEQ ID No.______).

The 5′ sequence is joined to a 3′ sequence comprising sequence that hybridizes to a portion of the polyA tail of mRNAs and at least one non-polyA nucleotide immediately upstream. Although the polyA-hybridizing sequence is typically a homopolymer of dT or dU, it need only contain a sufficient number of dT or dU bases to hybridize to polyA under the conditions employed. Both oligo-dT and oligo-dU primers have been used and give comparable results. Thus, other bases may be interspersed or concentrated, as long as hybridization is not impeded. Typically, 12 to 18 bases or 12 to 30 bases of dT or dU will be used. However, as one skilled in the art appreciates, the length need only be sufficient to obtain hybridization. The non-poly A⁺ nucleotide is A, C, or G, or a nucleotide derivative, such as inosinate. If one non-polyA nucleotide is used, then three oligonucleotide primers are needed to hybridize to all mRNAs. If two non-polyA nucleotides are used, then 12 primers are needed to hybridize to all mRNAs (AA, AC, AG, AT, CA, CC, CG, CT, GA, GC, GG, GT). If three non-poly A nucleotides are used then 48 primers are needed (3×4×4). Although there is no-theoretical upper limit on the number of non-polyA nucleotides, practical considerations make the use of one or two non-polyA nucleotides preferable.

For cDNA synthesis, the mRNAs are either subdivided into three (if one non-polyA nucleotide is used) or 12 (if two non-polyA nucleotides are used) fractions, each containing a single oligonucleotide primer, or the primers may be pooled and contacted with a mRNA preparation. Other subdivisions may alternatively be used. Briefly, first strand cDNA is initiated from the oligonucleotide primer by reverse transcriptase (RTase). As noted above, RASE may be obtained from numerous sources and protocols are well known. Second strand synthesis may be performed by RASE (Gubler and Hoffman, Gene 25: 263, 1983), which also has a DNA-directed DNA polymerase activity, with or without a specific primer, by DNA polymerase 1 in conjunction with RNaseH and DNA ligase, or other equivalent methods. The double-stranded cDNA is generally treated by phenol:chloroform extraction and ethanol precipitation to remove protein and free nucleotides.

Double-stranded cDNA is subsequently digested with an agent that cleaves in a sequence-specific manner. Such cleaving agents include restriction enzymes, chemical cleaving agents, triple helix, and any other cleaving agent available. Restriction enzyme digestion is preferred; enzymes that are relatively infrequent cutters (e.g., ≧5 bp recognition site) are preferred and those that leave overhanging ends are especially preferred. A restriction enzyme with a six base pair recognition site cuts approximately 8% of cDNAs, so that approximately 12 such restriction enzymes should be needed to digest every cDNA at least once. By using 30 restriction enzymes, digestion of every cDNA is assured.

The adapters for use in the present invention are designed such that the two strands are only partially complementary and only one of the nucleic acid strands that the adapter is ligated to can be amplified. Thus, the adapter is partially double-stranded (i.e., comprising two partially hybridized nucleic acid strands), wherein portions of the two strands are non-complementary to each other and portions of the two strands are complementary to each other. Conceptually, the adapter may be “Y-shaped” or “bubble-shaped.” When the 5′ region is non-paired, the 3′ end of other strand cannot be extended by a polymerase to make a complementary copy. The ligated adapter can also be blocked at the 3′ end to eliminate extension during subsequent amplifications. Blocking groups include dideoxynucleotides and other available blocking agents. In this type of adapter (“Y-shaped”), the non-complementary portion of the upper strand of the adapters is preferably a length that can serve as a primer for amplification. As noted above, the non-complementary portion of the lower strand need only be one base, however, a longer sequence is preferable (e.g., 3 to 20 bases; 3 to 15 bases; 5 to 15 bases, or 14 to 24 bases. The complementary portion of the adapter should be long enough to form a duplex under conditions of ligation.

For “bubble-shaped” adapters, the non-complementary portion of the upper strands is preferably a length that can serve as a primer for amplification. Thus, this portion is preferably 15 to 30 bases. Alternatively, the adapter can have a structure similar to the Y-shaped adapter, but has a 3′ end that contains a moiety that a DNA polymerase cannot extend from.

Amplification primers are also used in the present invention. Two different amplification steps are performed in the preferred aspect. In the first, the 3′ end (referenced to mRNA) of double stranded cDNA that has been cleaved and ligated with an adapter is amplified. For this amplification, either a single primer or a primer pair is used. The sequence of the single primer comprises at least a portion of the 5′ sequence of the oligonucleotide primer used for first strand cDNA synthesis. The portion need only be long enough to serve as an amplification primer. The primer pair consists of a first primer whose sequence comprises at least a portion of the 5′ sequence of the oligonucleotide primer as described above; and a second primer whose sequence comprises at least a portion of the sequence of one strand of the adapter in the non-complementary portion. The primer will generally contain all the sequence of the non-complementary potion, but may contain less of the sequence, especially when the non-complementary portion is very long, or more of the sequence, especially when the non-complementary portion is very short. In some embodiments, the primer will contain sequence of the complementary portion, as long as that sequence does not appreciably hybridize to the other strand of the adapter under the amplification conditions employed. For example, in one embodiment, the primer sequence comprises four bases of the complementary region to yield a 19 base primer, and amplification cycles are performed at 56° C. (annealing temperature), 72° C. (extension temperature), and 94° C. (denaturation temperature). In another embodiment, the primer is 25 bases long and has 10 bases of sequence in the complementary portion. Amplification cycles for this primer are performed at 68° C. (annealing and extension temperature) and 94° C. (denaturation temperature). By using these longer primers, the specificity of priming is increased.

The design of the amplification primers will generally follow well-known guidelines, such as average G-C content, absence of hairpin structures, inability to form primer-dimers and the like. At times, however, it will be recognized that deviations from such guidelines may be appropriate or desirable.

In instances where small numbers of cells are available for the initial RNA extraction, such as small numbers of stem cells, the preferred method of producing a gene expression profile comprises the following general steps. Total RNA is extracted from as few as 5000 stem cells. Using an oligo-dT primer, double stranded cDNA is synthesized and ligated to an adapter in accordance with the present invention. Using adapter primers, the cDNA is PCR amplified using the protocol of Baskaran and Weissman (1996) Genome Research 6(7): 633 and/or Liv et al. (1992) Methods of Enzymology. The original cDNA is therefore amplified several fold so that a large quantity of this cDNA is available for use in the display protocol according to the present invention. For the display, an aliquot of this cDNA is incubated with an anchored oligo-dT primer. In one method, this mixture is first heat denatured and then allowed to remain at 50° C. for 5 minutes to allow the anchor nucleotides of the oligo-dT primers to anneal. This provides for the synthesis of cDNA utilizing Klenow DNA polymerase. The 3′-end region of the parent cDNA (mainly the polyA region) that remains single stranded due to pairing and subsequent synthesis of cDNA by the anchored oligo-dT primer at the beginning of the polyA region, is removed by the 5′-3′ exonuclease activity of the T4 DNA polymerase. Following incubation of the cDNA with T4 DNA polymerase for this purpose, dNTPs are added in the reaction mixture so that the T4 DNA polymerase initiates synthesis of the DNA over the anchored oligo-dT primer carrying the heel. The net result of this protocol is that the cDNA with the 3′ heel is synthesized for display from the double stranded cDNA as the starting material, rather than RNA as the starting material as occurs in conventional 3′-end cDNA display protocol. The cDNA carrying the 3′-end heel is then subjected to restriction enzyme digestion, ligation, and PCR amplification followed by running the PCR amplified 3′-end restriction fragments with the Y-shaped adapter on a display gel. An alternate method is presented in Example 1.

After amplification, the lengths of the amplified fragments are determined. Any procedure that separates nucleic acids on the basis of size and allows detection or identification of the nucleic acids is acceptable. Such procedures include slab gel electrophoresis, capillary gel electrophoresis, 2-dimensional electrophoresis, high performance liquid chromatography, and the like.

Electrophoresis is technique based on the mobility of DNA in an electric field. Negatively charged DNA migrates towards a positive electrode at a rate dependent on their total charge, size, and shape. Most often, DNA is electrophoresed in agarose or polyacrylamide gels. For maximal resolution, polyacrylamide is preferred and for maximal linearity, a denaturant, such as urea is-present. A typical gel setup uses a 19:1 mixture of acrylamide:bisacrylarnide and a Tris-borate buffer. DNA samples are denatured and applied to the gel, which is usually sandwiched between glass plates. A typical procedure can be found in Sambrook et al. (Molecular Cloning: A Laboratory Approach, Cold Spring Harbor Press, NY, 1989) or Ausbel et al. (Current Protocols in Molecular Biology, Greene Publishing Co., NY, 1995). Variations may be substituted as long as sufficient resolution is obtained.

Capillary electrophoresis (CE) in its various manifestations (free solution, isotachophoresis, isoelectric focusing, polyacrylamide get micellar electrokinetic “chromatography”) allows high resolution separation of very small sample volumes. Briefly, in capillary electrophoresis, a neutral coated capillary, such as a 50 μm×37 cm column (eCAP neutral, Beckman Instruments, CA), is filled with a linear polyacrylamide (e.g., 0.2% polyacrylamide), a sample is introduced by high-pressure injection followed by an injection of running buffer (e.g., 1×TBE). The sample is electrophoresed and fragments are detected. An order of magnitude increase can be achieved with the use of capillary electrophoresis. Capillaries may be used in parallel for increased throughput (Smith et al. (1990) Nuc. Acids. Res. 18:4417; Mathies and Huang (1992) Nature 359:167). Because of the small sample volume that can be loaded onto a capillary, sample may be concentrated to increase level of detection. One means of concentration is sample stacking (Chien and Burgi (1992) Anal. Chem 64:489A). In sample stacking, a large volume of sample in a low concentration buffer is introduced to the capillary column. The capillary is then filled with a buffer of the same composition, but at higher concentration, such that when the sample ions reach the capillary buffer with a lower electric field, they stack into a concentrated zone. Sample stacking can increase detection by one to three orders of magnitude. Other methods of concentration, such as isotachophoresis, may also be used.

High-performance liquid chromatography (HPLC) is a chromatographic separation technique that separates compounds in solution. HPLC instruments consist of a reservoir of mobile phase, a pump, an injector, a separation column, and a detector. Compounds are separated by injecting an aliquot of the sample mixture onto the column. The different components in the mixture pass through the column at different rates due to differences in their partitioning behavior between the mobile liquid phase and the stationary phase. IP-RO-HPLC on non-porous PS/DVB particles with chemically bonded alkyl chains can also be used to analyze nucleic acid molecules on the basis of size (Huber et al. (1993) Anal. Biochem. 121:351; Huber et al. (1993) Nuc. Acids Res. 21:1061; Huber et al. (1993) Biotechniques 16:898).

In each of these analysis techniques, the amplified fragments are detected. A variety of labels can be used to assist in detection. Such labels include, but are not limited to, radioactive molecules (e.g., ³⁵S, ³²P, ³³P), fluorescent molecules, and mass spectrometric tags. The labels may be attached to the oligonucleotide primers or to nucleotides that are incorporated during DNA synthesis, including amplification.

Radioactive nucleotides may be obtained from commercial sources; radioactive primers may be readily generated by transfer of label from γ-³²P-ATP to a 5′-OH group by a kinase (e.g., T4 polynucleotide kinase). Detection systems include autoradiograph, phosphor image analysis and the like.

Fluorescent nucleotides may be obtained from commercial sources (e.g., ABI, Foster city, Calif.) or generated by chemical reaction using appropriately derivatized dyes. Oligonucleotide primers can be labeled, for example, using succinimidyl esters to conjugate to amine-modified oligonucleotides. A variety of florescent dyes may be used, including 6 carboxyfluorescein, other carboxyfluorescein derivatives, carboxyrhodamine derivatives, Texas red derivatives, and the like. Detection systems include photomultiplier tubes with appropriate wave-length filters for the dyes used. DNA sequence analysis systems, such as produced by ABI (Foster City, Calif.), may be used.

After separation of the amplified cDNA fragments, cDNA fragments which correspond to differentially expressed mRNA species are isolated, reamplified and sequenced according to standard procedures. For instance, bands corresponding the cDNA fragments can be cut from the electrophoresis gel, reamplified and subcloned into any available vector, including pCRscript using the PCR script cloning kit (Stratagene). The insert is then sequenced using standard procedures, such as cycle sequencing on an ABI sequencer (Foster City, Calif.).

An additional means of analysis comprises hybridization of the amplified fragments to one or more sets of oligonucleotides immobilized on a solid substrate. Historically, the solid substrate is a membrane, such as nitrocellulose or nylon. More recently, the substrate is a silicon wafer or a borosilicate slide. The substrate may be porous (Beattie et al. WO 95/11755) or solid. Oligonucleotides are synthesized in situ or synthesized prior to deposition on the substrate using standard procedures. Various chemistries are known for attaching oligonucleotides. Many of these attachment chemistries rely upon functionalizing oligonucleotides to contain a primary amine group. The oligonucleotides are arranged in an array form, such that the position of each oligonucleotide sequence can be determined.

The amplified fragments, which are generally labeled according to one of the methods described herein, are denatured and applied to the oligonucleotides on the substrate under appropriate salt and temperature conditions. In certain embodiments, the conditions are chosen to favor hybridization of exact complementary matches and disfavor hybridization of mismatches. Unhybridized nucleic acids are washed off and the hybridized molecules detected, generally both for position and quantity. The detection method will depend upon the label used. Radioactive labels, fluorescent labels and mass spectrometry label are among the suitable labels.

The present invention as set forth in the specific embodiments, includes methods to identify a therapeutic agent that modulates the expression of at least one stem cell gene associated with the differentiation, proliferation and/or survival of stem cells.

As an example, the method to identify an agent that modulates the expression of at least one stem cell gene associated with the differentiation of a stem cell population, comprises the steps of preparing a first gene expression profile of an undifferentiated stem cell population, preparing a second gene expression profile of a stem cell population at a defined stage of differentiation, treating said undifferentiated stem cell population with the agent, preparing a third gene expression profile of the treated stem cell population, and comparing the first, second and third gene expression profiles. Comparison of the three gene expression profiles for RNA species as represented by cDNA fragments that are differentially expressed upon addition of the agent to the undifferentiated stem cell population identifies agents that modulate the expression of a least one gene in undifferentiated stem cells that is associated with stem cell differentiation.

While the above methods for identifying a therapeutic agent comprise the comparison of gene expression profiles from treated and not-treated stem cells, many other variations are immediately envisioned by one of ordinary skill in the art. As an example, as a variation of a method to identify a therapeutic agent that modulates the expression of at least one stem cell gene associated with the differentiation, the second gene expression profile of a stem cell population at a defined stage of differentiation and the third gene expression profile of the treated stem cell population can each be independently normalized using the first gene expression profile prepared from the undifferentiated stem cell population. Normalization of the profiles can easily be achieved by scanning autoradiographs corresponding to each profile, and subtracting the digitized values corresponding to each band on the autoradiograph from undifferentiated stem cells from the digitized value for each corresponding band on autoradiographs corresponding to the second and third gene expression profiles. After normalization, the second and third gene expression profiles can be compared directly to detect cDNA fragments which correspond to mRNA species which are specifically expressed during differentiation of a stem cell population.

### SPECIFIC EMBODIMENTS

### Example 1

Production of Gene Expression Profiles Generated from cDNAs Made with RNA Isolated from Undifferentiated and Partially Differentiated Stem Cells.

Crude Marrow Preparation

Expression profiles of RNA expression levels from undifferentiated stem cells and stems cells at various levels of differentiation, including partially differentiated and terminally differentiated stem cells, offer a powerful means of identifying genes whose expression levels are associated with stem cell differentiation or proliferation. As an example, the production of expression profiles from murine lineage negative, rhodamine low, Hoechst low and rhodamine bright, Hoechst low hematopoietic precursor cells allows for the identification of mRNA species and their encoding genes whose expression levels are associated with stem cell differentiation.

Hoechst^{low}/Rhodamine^{low} hematopoietic stem cells were isolated by sacrificing 30 Balb/c female mice (6-12 weeks) and surgically removing the iliac crests, femurs and tibiae. The bones were cleaned and placed in 10 ml PBS/5% HI-FBS on ice. One tube was used for the bones from 10 mice. The bones were ground throughly with a pestle until completely broken. Following grinding, the supernatant was removed into a 50 ml conical tube through a 40 μM filer (Falcon #2340). 10 ml PBS/FBS was added to the mix and the supernatant removed. The supernatant was then centrifuged (1250 rpm) for 5-10 minutes. The supernatant which contains a high concentration of lipid was then decanted and discarded.

The cells were then pooled into 25 or 50 ml fresh PBS/FBS, and tiny bone fragments removed by settling. The cells were then counted in crystal violet. Cells were diluted and underlayed with LSM, centrifuged at 2000 rpm(1000×g) for 20 minutes. To harvest the buffy coat, the supernatant was removed to within 1 cm of the cells. The next 8-10 ml of medium and cells were harvested by swirling the media around in the tube to draw cells from all sides of the gradient. The cell volume was then brought up to 50 ml with PBS/FBS and spun at 1400 rpm 5-10 minutes.

Lineage Depletion

Cells were counted in Crystal Violet and resuspended in fresh PBS/FBS. Lineage-specific antibodies were added as follows:

| | |
|---|---|
| TER 119 | 0.1 μg/ml final concentration |
| B220 | 15 μl/10⁸ cells |
| Mac-1 | 15 μl/10⁸ cells |
| Gr-1 | 15 μl/10⁸ cells |
| Lyt-2 | 1/20 final dilution |
| L3T4 | 1/20 final dilution |
| Yw25.12.7 | 1/100 final dilution |

The cells were incubated on ice for 15 minutes, brought to a volume of 50 ml with PBS/FBS and collected at 1400 rpm for 5-10 minutes, and washed to remove unbound antibodies.

During the antibody binding step, Magnetic Beads(Dynabeads M-450) were prepared at a ratio of 5 beads/cell. The beads were coated with Sheep anti-Rat antibodies that bind to the lineage-specific antibodies, which are all of rat origin. When the beads are placed in a magnetic field, the Lin⁺ cells are removed. The resulting supernatant contains the Lin-population (granulocytes and lymphocyte populations will-be substantially depleted or absent after this step.)

Hoechst/Rhodamine Staining

Rhodamine 123 was added to a final concentration of 0.1 μg/ml, then incubated at 32° C. for 20 minutes in the dark. Without further manipulation or washing, HOECHST 33342 was added to a final concentration of 10 μM then incubated at 37° C. for an additional hour. The aliquot of crude marrow was brought to 0.5 ml with PBS/FBS and Hoechst to this cell preparation as well. The volume was brought to 50 ml with PBS/FBS, centrifuged at 1400 rpm for 5-10 minutes, supernatant discarded and cells resuspended to 2×10⁷ cells/ml. The rhodamine only and Hoechst Only/Crude Marrow were washed in parallel. These two populations were then resuspended in 0.5 ml PBS/FBS for flow cytometry analysis.

Total RNA was extracted from approximately 5000 stem cells. Using an oligo-dT primer, double stranded cDNA is synthesized and ligated to an adapter in accordance with the present invention. Using adapter primers, the cDNA is PCR amplified using the protocol of Baskaran and Weissman (1996) Genome Research 6(7): 633 and Lie et al., Methods of Enzymology,______. The original cDNA is therefore amplified several fold so that a large quantity of this cDNA is available for use in the display protocol according to the present invention.

Synthesis of cDNA for the gene expression profiles was performed as below:

Materials and Reagents

A microPoly(A)Pure mRNA Isolation kit (Ambion Inc.) was used for mRNA isolation. All the reagents for cDNA synthesis were obtained from Life Technologies Inc. Klentaq1 DNA polymerase (25 U/μl) was from Ab peptides Inc. Native Pfu DNA polymerase (2.5 U/μl) was purchased from Stratagene Inc. Betaine monohydrate was from Fluka BioChemica and dimethylsulfoxide (DMSO) was from Sigma Chemical Company. Deoxynucleoside triphophates (dNTPs, 100 mM) and bovine serum albumin (BSA, 10 mg/ml) were purchased from New England Biolabs, Inc. Qiaquick PCR purification kit (Qiagen) was used to purify the amplified PCR products. The oligonucleotides used in the Examples were synthesized and gel purified in the DNA synthesis laboratory (Department of Pathology, Yale University School of Medicine, New Haven, Conn.).

TABLE 1
Sequences of oligonucleotides.

| | |
|---|---|
| T₇-SalI-oligo-d(T)V | 5′-ACG TAA TAC GAC TCA CTA TAG |
| | GGC GAA TTG GGT CGA C-d(T)₁₈V- |
| | 3′, where V = A, C, G |
| anti-NotI Long | 5′-CTT ACA GCG GCC GCT TGG |
| | ACG-3′ |
| NotI Short | 5′-AGC GGC CGC TGT AAG-3′ |
| NotI/RI primer | 5′-GCG GAA TTC CGT CCA AGC GGC |
| | CGC TGT AAG-3′ |

Methods
I. Preparation of mRNA

MicroPoly(A)Pure mRNA isolation kit was used for the isolation of Poly(A)⁺ RNA following the kit instructions. mRNA from a small number of mouse hematopoietic cells (5,000-10,000 cells) was extracted, eluted from the column, and precipitated by adding 0.1 volume of 5 M ammonium acetate and 2.5 volumes of chilled ethanol with 2 μg glycogen as carrier. The tubes were left at −20° C. overnight. The pellets were collected by centrifugation at top speed for 30 minutes, washed with 70% ethanol and air-dried at room temperature. The pellets were resuspended in 10 μl H₂O/0.1 mM EDTA solution. We observed that the dissolved mRNA solution was cloudy due to the leaching of column materials, therefore the samples were centrifuged at 4° C. for 5 minutes. The supernatant was collected for further use.

II. cDNA Synthesis

First Strand cDNA Synthesis

The cDNA synthesis reaction (final reaction volume is 20 μl) was carried out as described in the instruction manual (Superscript Choice System) provided by Life Technologies Inc. For the first strand cDNA synthesis, mRNA (10 μl) isolated from a small number of cells was annealed with 200 ng (1 μl) of T₇-SalI-oligo-d(T)V-primer (see Table- 1) in a 0.5-ml micro centrifuge tube (no stick, USA Scientific Plastics) by heating the tubes at 65° C. for 5 minutes, followed by quick chilling on ice for 5 minutes. This step was repeated once and the contents were collected at the bottom of the tube by a brief centrifugation. The following components were added to the primer annealed mRNA on ice prior to initiating the reaction, 1 μl of 10 mM dNTPs, 4 μl of 5×first strand buffer [250 mM Tris-HCl (pH 8.3), 375 mM KCl, 15 mM MgCl₂], 2 μl of 100 mM DTT and 1 μl of RNase Inhibitor (40 U/μl). All the contents were mixed gently and the tubes were pre-warmed at 45° C. for 2 minutes. The cDNA synthesis was initiated by adding 200 units (1 μl) of Superscript II Reverse Transcriptase and the incubation continued at 45° C. for 1 hour.

Second Strand cDNA Synthesis

At the end of first strand cDNA synthesis, the tubes were kept on ice. Second strand cDNA synthesis reaction (final volume is 150 μl) was set up in the same tube on ice by adding 91 μl of nuclease free water, 30 μl of 5×second strand buffer [100 mM Tris-HCl (pH 6.9), 23 mM MgCl₂, 450 mM KCl, 0.75 mM (β-NAD⁺ and 50 mM ammonium sulfate], 3 μl of 10 mM dNTPs, 1 μl of E. coli DNA ligase (10 U/μl), 4 μl of E. coli DNA polymerase I (10 U/μl) and 1 μl of E. coli RNase H (2 U/μl). The contents were mixed gently and the tubes were incubated at 16° C. for 2 hours. Following the incubation, the tubes were kept on ice, 2 μl of T₄ DNA polymerase (3 U/μl) was added and the incubation was continued for another 5 minutes at 16° C. The reaction was stopped by the addition of 10 μl of 0.5 M EDTA (pH 8.0) and extracted once with equal volume of phenol: chloroform 1:1 (v/v) and once with chloroform. The aqueous phase was then transferred to a new tube and precipitated by adding 0.5 volumes of 7.5 M ammonium acetate (pH 7.6), 2 μg of glycogen (as carrier) and 2.5 volumes of chilled ethanol. The samples were left at −20° C. for overnight and the cDNA pellets were collected by centrifugation at top speed for 20 minutes. The pellets were washed once with 70% ethanol, air-dried and dissolved in 14 μl of nuclease free water.

As the amount of cDNA derived from a small number of cells may be low, it may be necessary to amplify the cDNA for further analysis. To uniformly amplify the cDNA, an adaptor (NotI adaptor) was first ligated to both ends of the cDNA. Following adaptor ligation, the cDNAs were amplified with NotI/RI primer (see table 1), by a modified PCR method using betaine and DMSO.

Ligation of cDNA with NotI Adaptor

Preparation of NotI adaptor: The NotI adaptor was prepared by annealing NotI-short and anti-NotI-long oligonucleotides (see Table 1). The anti-NotI-long oligonucleotide was phosphorylated to ensure that both the adaptor oligonucleotides are ligated to the cDNA. 1 μg of anti-NotI-long was mixed with 1 μl of 10×T₄ polynucleotide kinase buffer [700 mM Tris-HCl (pH 7.6), 100 mM MgCl₂ and 50 mM DTT], 1 μl of 10 mM adenosine triphosphate (ATP), adjusted the volume to 9 μl with water and the reaction was initiated by adding 1 μl of T₄ polynucleotide kinase (10 U/μl). The tubes were incubated at 37° C. for 30 minutes and then the enzyme was inactivated at 65° C. for 20 minutes. The annealing was carried out by adding the following components to the above phosphorylated anti-NotI-long: 1 μg of NotI-short, 2 μl of 10×oligo annealing buffer [100mM Tris-HCl (pH 8.0), 10 mM EDTA (pH 8.0) and 1 M NaCl] and water to adjust the final volume to 20 μl. The sample was heated at 65° C. for 10 minutes and allowed to cool down to room temperature. The annealed adaptor was stored at −20° C.

Ligation of cDNA with annealed NotI adaptor: To set up this reaction, 14 μl of cDNA was mixed with 100 ng of annealed NotI adaptor in a 0.5-ml micro centrifuge tube. To this mixture 2 μl of 10×T₄ DNA ligase buffer [500 mM Tris-HCl (pH 7.8),100 mM MgCl₂, 100 mM DDT, 10 mM ATP and 250 mg/ml BSA] was added and adjusted the volume with water to 18 μl and mixed gently. The reaction was initiated by adding 2 μl of T₄ DNA ligase (400 U/μl) and incubated at 16° C. overnight.

III. cDNA Amplification

A modified betaine-DMSO PCR method (Baskaran et al. (1996)) Genome Research 6:633) was used to uniformly amplify the cDNA with different GC content. This method uses the LA system, which combines a highly thermostable form of Taq DNA polymerase (Klentaq1, which is devoid of 5′-exonuclease activity) and a proofreading enzyme (Pfu DNA polymerase, which has 3′-exonuclease activity). The LA16 enzyme consists of 1 part of Pfu DNA polymerase and 15 parts of KlenTaq1 DNA Polymerase (v/v). The NotI adaptor-ligated cDNA was diluted 10 fold with water. 2 μl of this diluted cDNA was used as the template for PCR. The PCR reaction (50 μl final volume) was set up with the following components: 5 μl of 10×PCR buffer [200 Mm Tris-HCl (pH 9.0), 160 mM ammonium sulfate and 25 mM MgCl₂], 16 μl of water, 0.8 μl of BSA (10 mg/ml), 1 μl of NotI/RI PCR primer (100 ng/ul), 5 μl of 50% DMSO (v/v), 15 μl of 5 M Betaine and 0.2 μl of LA16 enzyme. These components were mixed gently on ice and then heated to 95° C. for 15 seconds on a PCR machine, and held at 80° C. while 5 μl of 2 mM dNTPs were added to start the reaction. The PCR conditions were as follows: Stage 1: 95° C. for 15 seconds, 55° C. for 1 minute, 68° C. for 5 minutes, 5 cycles. Stage 2: 95° C. for 15 seconds, 60° C. for 1 minute, 68° C. for 5 minutes, 15 cycles.

After amplification, cDNA was purified with the Qiaquick PCR purification kit (following the instructions provided by the supplier). The purified cDNA was eluted in the desired volume of water.

Gene expression profiles were prepared from the purified cDNA as previously described by Prashar et al. in WO 97/05286 and in Prashar et al. (1996) Proc. Natl. Acad. Sci. USA 93:659-663. Briefly, the adapter oligonucleotide sequences were CTTACAGCGGCCGCTTGGACG, GAATGTCGCCGGCGA or alternatively, A1 (TAGCGTCCGGCGCAGCGACGGCCAG) and A2 (GATCCTGGCCGTCGGCTGTCTGTCGGCGC). When A1/A2 were used, one microgram of oligonucleotide A2 was first phosphorylated at the 5′ end using T4 polynucleotide kinase (PNK). After phosphorylation, PNK was heated denatured, and 1 μg of the oligonucleotide A1 was added along with 10×annealing buffer (1 M NaCl/100 mM Tris-HCl, pH 8.0/10 mM EDTA, pH8.0) in a final vol of 20 μl. This mixture was then heated at 65° C. for 10 min followed by slow cooling to room temperature for 30 min, resulting in formation of the Y adapter at a final concentration of 100 ng/μl. About 20 ng of the cDNA was digested with 4 units of a restriction enzyme such as ClaI, Bgl II, etc. in a final vol of 10 μl for 30 min at 37° C. Two microliters (≈4 ng of digested cDNA) of this reaction mixture was then used for ligation to 100 ng (≈50-fold) of the Y-shaped adapter in a final vol of 5 μl for 16 hr at 15° C. After ligation, the reaction mixture was diluted with water to a final vol of 80 μl (adapter ligated cDNA concentration, ≈50 pg/μl) and heated at 65° C. for 10 min to denature T4 DNA ligase, and 2-μl aliquots (with ≈100 pg of cDNA) were used for PCR.

The following sets of primers were used for PCR amplification of the adapter ligated 3′-end cDNAs: GCGGAATTCCGTCCAAGCGGCCGCTGTAAG or alternatively, RP 5.0 (CTCTCAAGGATCTTACCGCTT₁₈AT), RP 6.0 (TAATACCGCGCCACATAGCAT₁₈CG), or RP 9.2 (CAGGGTAGACGACGCTACGCT₁₈GA) were used as 3′ primer while A1.1 (TAGCGTCCGGCGCAGCGAC) served as the 5′ primer. To detect the PCR products on the display gel, 24 pmol of oligonucleotide A1.1 was 5′-end-labeled using 15 μl of [γ-³²P]ATP (Amersham; 3000 Ci/mmol) and PNK in a final volume of 20 μl for 30 min at 37° C. After heat denaturing PNK at 65° C. for 20 min, the labeled oligonucleotide was diluted to a final concentration of 2 μM in 80 μl with unlabeled oligonucleotide A1.1. The PCR mixture (20 μl) consisted of 2 μl (≈100 pg) of the template, 2 μl of 10×PCR buffer (100 mM Tris·HCl, pH 8.3/500 mM KCl), 2 μl of 15 mM MgCl₂ to yield 1.5 mM final Mg²⁺ concentration optimum in the reaction mixture, 200 μM dNTPs, 200 nM each 5′ and 3′ PCR primers, and 1 unit of Amplitaq. Primers and dNTPs were added after preheating the reaction mixture containing the rest of the components at 85° C. This “hot start” PCR was done to avoid artefactual amplification arising out of arbitrary annealing of PCR primers at lower temperature during transition from room temperature to 94° C. in the first PCR cycle. PCR consisted of 28-30 cycles of 94° C. for 30 sec, 50° C. for 2 min, and 72° C. for 30 sec. A higher number of cycles resulted in smeary gel patterns. PCR products (2.5 μl) were analyzed on 6% polyacrylamide sequencing gel. For double or multiple digestion following adapter ligation, 13.2 μl of the ligated cDNA sample was digested with a secondary restriction enzyme(s) in a final vol of 20 μl. From this solution, 3 μl was used as template for PCR. This template vol of 3 μl carried≈100 pg of the cDNA and 10 mM MgCl₂ (from the 10×enzyme buffer), which diluted to the optimum of 1.5 mM in the final PCR vol of 20 μl. Since Mg²⁺ comes from the restriction enzyme buffer, it was not included in the reaction mixture when amplifying secondarily cut cDNA. Bands may then be extracted from the display gels as described by Liang et al. (1995 Curr. Opin. Immunol. 7:274-280), reamplified using the 5′ and 3′ primers, and subcloned into pCR-Script with high efficiency using the PCR-Script cloning kit from Stratagene. Plasmids were sequenced by cycle sequencing on an ABI automated sequencer.

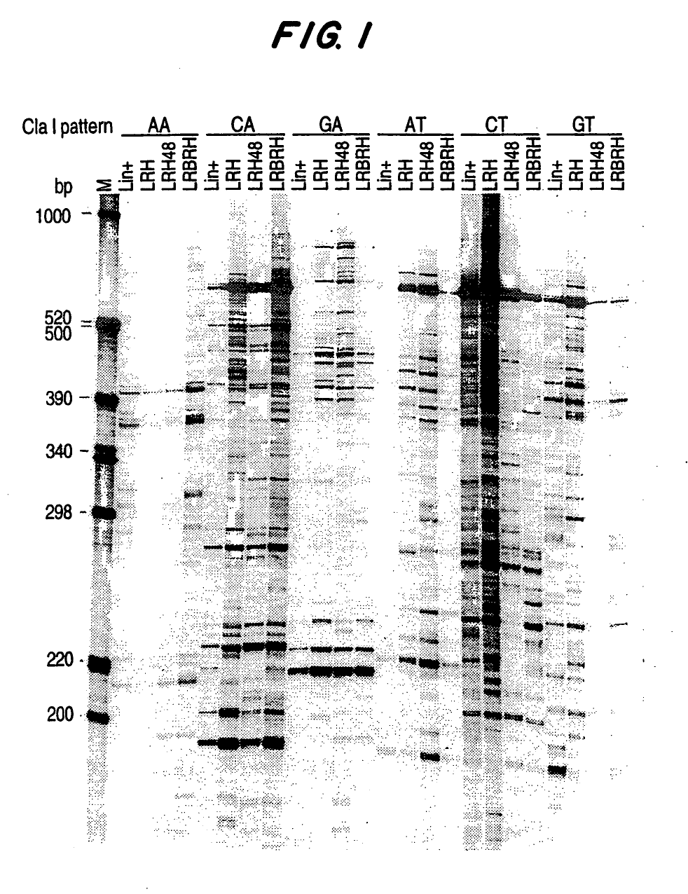

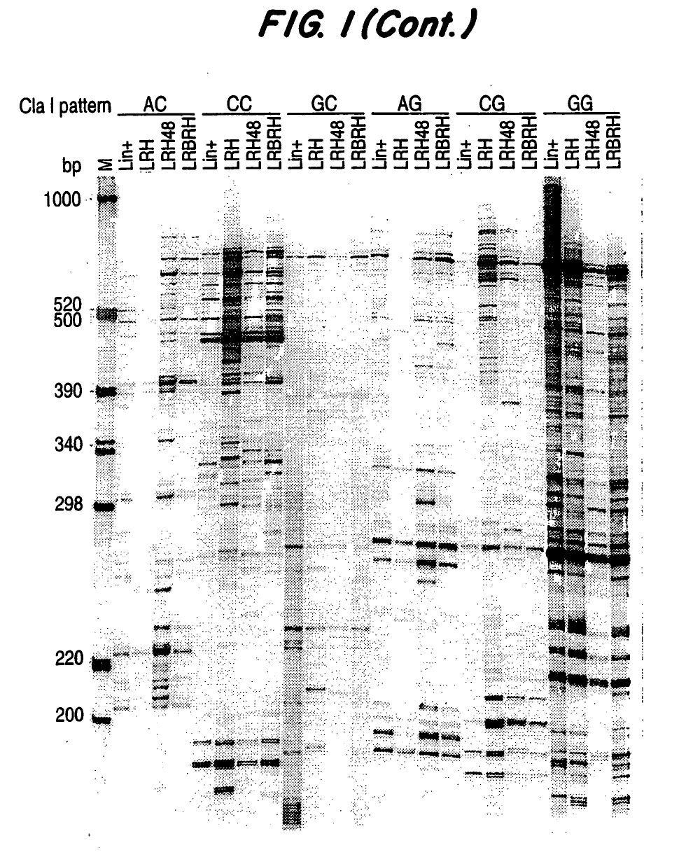

FIG. 1 presents an autoradiogram of the gene expression profiles generated from cDNAs made with RNA isolated from Lin⁺, LRH, LRH48 and LRBRH cells. All possible 12 anchoring oligo d(T)n1, n2 were used to generate a complete expression profile for the enzyme ClaI.

Table 2 presents the sequences of numerous differentially expressed bands from expression profiles made from LIN⁺, LRH, LRH48 and LRBRH.

Table 3 presents the expression patterns of the differentially expressed bands set forth in Table 2. The band fragment length (size) in Table 3 is the length before unwanted terminal sequences were removed. Table 3 also presents the results of a GenBank Search and analysis of the sequences of Table 2.

Summary of Known Genes from Mouse HSC Differential Display (I)

| | | | | | [symbol] | | | | |
|---|---|---|---|---|---|---|---|---|---|
| Items No. | Size (bp) | Enzyme | NIN2 (oligo-dT) | Poly(A) Sign | [symbol] | [symbol] | [symbol] | [symbol] | [symbol] |
| HSC-DD-006 | 213 | Bgl II | AC | fair | 0 | 3+ | / | + | [symbol] |
| HSC-DD-285 | 158 | Xba I | GG | good | ± | + | + | ± | human homeobox gene regulator |
| HSC-DD-0078 | 213 | Bgl II | AC | fair | ± | 2+ | / | ± | human zinc finger protein 10 |
| HSC-DD-238 | 363 | Xba I | AG | good | 3+ | 0 | 3+ | 3+ | mouse cell division control protein 19 |
| HSC-DD-206 | 123 | Xba I | AC | good | 3+ | 0 | 2+ | + | human HS1 [symbol] protein |
| HSC-DD-214 | 192 | Xba I | AC | fair | ± | 2+ | 0 | 3+ | mouse plan-1 prolo-oncogene |
| HSC-DD-035 | 151 | Bgl I | AC | fair | ± | 2+ | / | + | mouse thyroid hormone receptor |
| HSC-DD-129 | 234 | Cla I | AC | poor | 0 | 3+ | 0 | 0 | mouse [symbol] 1.4.5-trisphosphate receptor |
| HSC-DD-040 | 220 | Bgl II | AC | fair | + | 2+ | / | 0 | mouse G protein beta-36 subunit |
| HSC-DD-011 | 173 | Bgl II | AC | good | ± | ± | / | 2+ | mouse ras-related YPT1 protein |
| HSC-DD-121 | 186 | Cla I | CT | poor | 0 | 3+ | ± | ± | human TBP-associated factor 170 |
| HSC-DD-015B | 133 | Bgl II | AG | poor | 0 | 3+ | / | + | mouse HMG1-related DNA binding protein |
| HSC-DD-039 | 206 | Bgl II | AC | fair | 2+ | 4+ | / | 4+ | mouse TAX responsive element binding |
| | | | | | | | | | protein 107 |
| HSC-DD-042 | 235 | Bgl II | AC | fair | ± | 0 | / | + | mouse retinoblastoma binding protein |
| | | | | | | | | | isoform III |
| HSC-DD-256 | 272 | Xba II | AA | poor | 0 | 2+ | ± | 0 | Rat antrogen-binding protein |
| HSC-DD-045 | 270 | Bgl II | AC | good | ± | 2+ | / | ± | similar to Rat cca2 |
| HSC-DD-068 | 164 | Cla I | AC | fair | + | 4+ | 4+ | 4+ | mouse [symbol] mRNA |
| HSC-DD-143 | 350 | Cla I | AG | fair | ± | 2+ | ± | ± | similar to human mamd |
| HSC-DD-263 | 292 | Xba I | AT | good | 0 | 2+ | ± | 0 | mouse [symbol] 5 |
| HSC-DD-239 | 156 | Xba I | CA | good | ± | 3+ | 3+ | + | human CD9 |
| HSC-DD-261 | 115 | Xba I | AA | good | 0 | + | 0 | 0 | mouse [symbol] [symbol] |
| HSC-DD-02 [symbol] | 95 | Bgl II | AC | good | + | 4+ | / | + | mouse [symbol] [symbol] TCP-1 [symbol] subunit |
| HSC-DD-021 | 143 | Bgl II | AG | [symbol] | ± | + | / | 2+ | mouse [symbol] |
| HSC-DD-025 | 326 | Bgl II | AG | poor | ± | 2+ | / | 2+ | mouse [symbol] I |

Summary of Known Genes from Mouse HSC Differential Display (II)

| | | | | | [symbol] | | | | |
|---|---|---|---|---|---|---|---|---|---|
| Items No. | Size (bp) | Enzyme | NIN2 (oligo-dT) | Poly(A) Sign | [symbol] | [symbol] | [symbol] | [symbol] | [symbol] |
| HSC-DD-077 | 203 | Cla I | AC | good | + | 2+ | 2+ | 3+ | Rat matrin cyclophilin |
| HSC-DD-200 | 450 | Cla I | AA | fair | + | ± | 2+ | + | mouse G-utrophin |
| HSC-DD-245 | 272 | Xba I | CA | fair | 3+ | ± | 3+ | 2+ | rat basement membrane-associated |
| | | | | | | | | | chondroilin |
| HSC-DD-226 | 387 | Xba I | AC | good | ± | 3+ | ± | 0 | mouse cycloplasmic g-actin |
| HSC-DD-182 | 149 | Cla I | GC | poor | ± | 3+ | ± | + | mouse A-X actin |
| HSC-DD-089 | 364 | Cla I | AC | poor | + | 3+ | 2+ | + | mouse TIE receptor tyrosine kinase |
| HSC-DD-151 | 424 | Cla I | GA | good | 0 | + | 2+ | ± | rat elk, brain-specific receptor tyrosine |
| | | | | | | | | | kinase |
| HSC-DD-013 | 248 | Bgl II | AC | fair | ± | 2+ | / | 3+ | mouse hexokinase |
| HSC-DD-029 | 103 | Bgl II | AC | fair | 0 | + | / | 0 | mouse brain [symbol] tyrosine kinase |
| HSC-DD-034 | 140 | Bgl II | AC | fair | 0 | 2+ | / | 2+ | mouse spermine synthase |
| HSC-DD-082B | 244 | Cla I | AC | fair | + | 4+ | 2+ | 2+ | mouse stearoyl-CoA desalurase (SCD2) |
| HSC-DD-084 | 261 | Cla I | AC | good | ± | + | ± | 2+ | mouse antioxidant enzyme AOE 372 |
| HSC-DD-128 | 189 | Cla I | AC | fair | 0 | 3+ | 3+ | ± | mouse casein kinase II beta chain |
| HSC-DD-140 | 229 | Cla I | AG | good | ± | 0 | 0 | + | mouse creatine kinase B |
| HSC-DD-148 | 313 | Cla I | GA | good | + | + | 2+ | ± | human esterase D |
| HSC-DD-176 | 470 | Cla I | CG | fair | ± | 3+ | + | 0 | mouse putative E1-E2 ATPase |
| HSC-DD-178 | 130 | Cla I | GC | good | ± | 3+ | 0 | + | mouse aspartate aminotransferase |
| HSC-DD-180 | 142 | Cla I | GC | good | + | + | 0 | + | mouse tyrosyprotein sulfotransferase-1 |
| HSC-DD-186 | 252 | Cla I | GC | poor | ± | + | 2+ | 2+ | mouse ubiquitin-conjugating enzyme E214K |
| HSC-DD-191 | 136 | Cla I | AA | fair | 0 | ± | 3+ | 2+ | mouse b-1,4-galactosyltransferase |
| HSC-DD-158 | 391 | Cla I | GT | fair | + | 3+ | 0 | + | spermophilus tridecenlinealus 26s |
| | | | | | | | | | proteasome |
| HSC-DD-099 | 265 | Cla I | CC | fair | ± | 3+ | 0 | ± | mouse proteasome [symbol] chain precursor |
| HSC-DD-222 | 270 | Xba I | AC | good | 0 | 2+ | 3+ | + | Rat 3-hydrolase butyrate |
| HSC-DD-104 | 368 | Cla I | CC | fair | 0 | ± | 0 | ± | human copper chaperone for superoxide |
| | | | | | | | | | dismulase |
| HSC-DD-172 | 365 | Cla I | CG | fair | ± | 3+ | 2+ | 0 | mouse Ercc-4 DNA repair gene |
| HSC-DD-169 | 223 | Cla I | CG | fair | ± | ± | 2+ | 0 | C [symbol] guseus nucleotide excision repair protein |
| HSC-DD-003A | 148 | Bgl II | AC | poor | 0 | + | / | ± | human G rich sequence factor |

Summary of Known Genes from Mouse HSC Differential Display (III)

| | | | | | Expression pattern | | | | |
|---|---|---|---|---|---|---|---|---|---|
| Item No. | Size (bp) | Enzyme | NIN2 (oligo-dT) | Poly(A) Sign | Lin+ | LRII | LRII48 | LRBR11 | Gene Bank Search & Analysis |
| HSC-DD-092 | 118 | Cla I | CC | fair | + | 3+ | ± | + | mouse elongation factor 1-a |
| HSC-DD-288 | 480 | Xba I | GC | fair | ± | + | + | ± | human elongation factor-1-delta |
| HSC-DD-114 | 267 | Cla I | CA | poor | ± | + | ± | + | Rat elongation factor-1-alpha |
| HSC-DD-213 | 178 | Xba I | AC | fair | ± | 3+ | + | + | human splicing factor (SFRS7) |
| HSC-DD-155 | 198 | Cla I | GT | fair | 0 | 2+ | + | 0 | mouse transcription elongation factor |
| | | | | | | | | | S-II-T1 |
| HSC-DD-212 | 162 | Xba I | AC | poor | 0 | 3+ | ± | 0 | mouse translation initiation factor 4E |
| HSC-DD-090 | 375 | Cla I | AC | fair | ± | 3+ | 3+ | + | mouse protein synthesis elongation factor |
| HSC-DD-173 | 367 | Cla I | CG | fair | ± | 3+ | + | 0 | mouse protein synthesis elongation factor |
| | | | | | | | | | Tu |
| HSC-DD-249 | 304 | Xba I | CA | poor | 4+ | + | 4+ | 4+ | rat histone macroH2A1.2 |
| HSC-DD-250 | 356 | Xba I | CA | good | + | 2+ | 3+ | 2+ | mouse MER9 processed pseudogene |
| HSC-DD-108 | 281 | Cla I | GG | good | + | 2+ | + | 2+ | mouse heat shock protein 70 |
| HSC-DD-116 | 326 | Cla I | CA | fair | ± | 2+ | 0 | 2+ | mouse 84 kD heat shock protein |
| HSC-DD-166 | 587 | Cla I | AT | good | ± | 2+ | 3+ | + | mouse heat shock protein 70 cognate |
| HSC-DD-184 | 196 | Cla I | GC | fair | ± | 2+ | 0 | ± | mouse breast heat shock protein 73 |
| HSC-DD-101 | 331 | Cla I | CC | fair | + | 3+ | 0 | ± | mouse MHC locus II region |
| HSC-DD-017 | 215 | Bgl II | AG | good | 0 | 4+ | / | 0 | mouse MHC class III region |
| HSC-DD-026 | 505 | Bgl II | AG | fair | 2+ | 4+ | / | 4+ | mouse ribosomal protein S4 |
| HSC-DD-064 | 146 | Cla I | AC | good | 2+ | 2+ | 2+ | 3+ | mouse ribosomal protein S12 |
| HSC-DD-066 | 150 | Cla I | AC | good | 2+ | 3+ | 2+ | 2+ | mouse ribosoaml protein S20 |
| HSC-DD-041 | 226 | Bgl II | AC | good | + | 3+ | / | 3+ | mouse ribosomal protein L7 |
| HSC-DD-111 | 161 | Cla I | CA | fair | ± | + | ± | + | rat ribosomal protein L23a |
| HSC-DD-0288 | 100 | Bgl II | AC | fair | + | 4+ | / | + | mouse LINE-1/L1 element |
| HSC-DD-142 | 267 | Cla I | AG | fair | ± | 2+ | ± | ± | mouse L1Md A13 repetitive sequence |
| HSC-DD-095 | 210 | Cla I | CC | fair | ± | 2+ | ± | ± | mouse mitochondrial [symbol] 2S ribosomal RNA |

As is apparent to one of ordinary skill in the art, this same procedure can be used to identify stem cells genes whose expression levels are associated with stem cell proliferation, dedicated differentiation and survival.

### Example 2

Method to Identify a Therapeutic Agent that Modulates the Expression of at Least One Stem Cell Gene Associated with the Differentiation Process of a Stem Cell Population.

The methods set forth in Example 1 offer a powerful approach for identifying therapeutic agents that modulate the expression of at least one stem cell gene associated with the differentiation process of a stem cell population. For instance, gene expression profiles of undifferentiated stem cells and partially differentiated or terminally differentiated stem cells are prepared as set forth in Example 1. A profile is also prepared from an undifferentiated stem cell sample that has been exposed to the agent to be tested. By examining for differences in the intensity of individual bands between the three profiles, agents which up or down regulate genes associated with the differentiation process of a stem cell population are identified.

### Example 3

Method to Identify a Therapeutic Agent that Modulates the Expression of at Least One Stem Cell Gene Associated with the Proliferation of a Stem Cell Population.

The methods set forth in Example 1 offer a powerful approach for identifying therapeutic agents that modulate the expression of at least one stem cell gene associated with the proliferation of a stem cell population. For instance, gene expression profiles of undifferentiated stem cells and actively proliferating stem cells are prepared as set forth in Example 1. A profile is also prepared from an undifferentiated stem cell sample that has been exposed to the agent to be tested. By examining for differences in the intensity of individual bands between the three profiles, agents which up or down regulate genes associated with the proliferation of a stem cell population are identified.

As is apparent to one of ordinary skill in the art, this same procedure can be used to identify stem cells genes whose expression levels are associated with stem cell dedicated differentiation and survival.

### Example 4

Production of Solid Support Compositions Comprising Groupings of Nucleic Acids or Nucleic Acid Fragments that Correspond to Genes Whose Expression Levels are Associated with the Differentiation, Proliferation, Dedicated Differentiation or Survival of Stem Cells.

As set forth in Example 1, expression profiles prepared from stem cells at different stages of differentiation, from proliferating stem cells, from stem cells that are dedicated to a differentiation pathway and from stem cells resistant to apoptosis (which may be linked to increased survival) provide a means to identify genes whose expression levels are associated with stem cell differentiation, proliferation, dedicated differentiation and survival, respectively.

Solid supports can be prepared that comprise immobilized representative groupings of nucleic acids or nucleic acid fragments corresponding to the genes from stem cells whose expression levels are modulated during stem cell differentiation, proliferation, dedicated differentiation and survival. For instance, representative nucleic acids can be immobilized to any solid support to which nucleic acids can be immobilized, such as positively charged nitrocellulose or nylon membranes (see Sambrook et al. (1989) Molecular Cloning: a Laboratory Manual, 2nd Ed., Cold Spring Harbor Laboratory) as well as porous glass wafers such as those disclosed by Beattie (WO 95/11755). Nucleic acids are immobilized to the solid support by well established techniques, including charge interactions as well as attachment of derivatized nucleic acids to silicon dioxide surfaces such as glass which bears a terminal epoxide moiety. At least one species of nucleic acid molecule, or fragment of a nucleic acid molecule corresponding to the genes from stem cells whose expression levels are modulated during stem cell differentiation, proliferation, dedicated differentiation and survival may be immobilized to the solid support. A solid support comprising a representative grouping of nucleic acids can then be used in standard hybridization assays to detect the presence or quantity of one or more specific nucleic acid species in a sample (such as a total cellular mRNA sample or cDNA prepared from said mRNA) which hybridize to the nucleic acids attached to the solid support. Any hybridization methods, reactions, conditions and/or detection means can be used, such as those disclosed by Sambrook et al. (1989) Molecular Cloning: a Laboratory Manual, 2nd Ed., Cold Spring Harbor Laboratory, Ausbel et al. (1987) Current Protocols in Molecular Biology, Greene Publishing and Wiley-Interscience. N.Y. or Beattie in WO 95/11755.

One of ordinary skill in the art may determine the optimal number of genes that must be represented by nucleic acid fragments immobilized on the solid support to effectively differentiate between samples that are at the various stages of stem cell differentiation, including terminal differentiation, proliferating stem cells, stem cells dedicated to a given differentiation pathway and/or stem cells with increased survival rates. Preferably, at least about 5, 10, 20, 50, 100, 150, 200, 300, 500, 1000 or more preferably, substantially all of the detectable mRNA species in a cell sample or population will be present in the gene expression profile or array affixed to a solid support. More preferably, such profiles or arrays will contain a sufficient representative number of mRNA species whose expression levels are modulated under the relevant differentiation process, disease, screening, treatment or other experimental conditions. In most instances, a sufficient representative number of such mRNA species will be about 1, 2, 5, 10, 15, 20, 25, 30, 40, 50, 50-75 or 100 in number and will be represented by the nucleic acid molecules or fragments of nucleic acid molecules immobilized on the solid support. For example, nucleic acids encoding all or a fragment of one or more of the known genes or previously reported ESTs that are identified in Tables 2 and 3 may be so immobilized. Additionally, the skilled artisan may select nucleic acids encoding the protein cell surface markers discussed above at page 8 (i.e., CD 34) in order to help identify the particular stage of differentiation of a given stem cell population and to identify agents that are involved in promoting such differentiation. The skilled artisan will be able to optimize the number and particular nucleic acids for a given purpose, i.e., screening for modulating agents, identifying activated stem cells, etc.

In general, nucleic acid fragments comprising at least one of the sequences or part of one of the sequences of Table 2 can be used as probes to screen nucleic acid samples from cell populations in hybridization assays. Alternatively, nucleic acid fragments derived from the identified genes in Table 3 which correspond to the sequences of Table 2 may be employed as probes. To ensure specificity of a hybridization assay using probe derived from the sequences presented in Table 2 or the genes of Table 3, it is preferable to design probes which hybridize only with target nucleic acid under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the sequences of Table 2 or the genes of Table 3 through methods known in the art. For instance, the G+C content of the probe and the probe length can affect probe binding to its target sequence. Methods to optimize probe specificity are commonly available in Sambrook et al. (Molecular Cloning: A Laboratory Approach, Cold Spring Harbor Press, NY, 1989) or Ausubel et al. (Current Protocols in Molecular Biology, Greene Publishing Co., NY, 1995). Any available format may be used in designing hybridization assays, including immobilizing the probes to a solid support or immobilizing the cellular test sample nucleic acids to a solid support.

It should be understood that the foregoing discussion and examples merely present a detailed description of certain preferred embodiments. It therefore should be apparent to those of ordinary skill in the art that various modifications and equivalents can be made without departing from the spirit and scope of the invention. All documents, patents and references, including provisional patent application 60/056,861, referred to throughout this application are herein incorporated by reference.

What is claimed is:

1. A method to identify an agent that modulates the expression of at least one stem cell gene associated with the differentiation process of a stem cell population, comprising the steps of:
preparing a first gene expression profile of an undifferentiated stem cell population;
preparing a second gene expression profile of a stem cell population at a defined stage of differentiation;
treating said undifferentiated stem cell population with the agent;
preparing a third gene expression profile of the treated undifferentiated stem cell population;
comparing the first, second and third gene expression profiles; and
identifying an agent that modulates the expression of a least one gene in undifferentiated stem cells that is associated with stem cell differentiation.

2. A method to identify an agent that modulates the expression of at least one stem cell gene associated with the proliferation of a stem cell population, comprising the steps of:
preparing a first gene expression profile of a non-proliferating stem cell population;
preparing a second gene expression profile of a proliferating stem cell population;
treating the non-proliferating stem cell population with the agent;
preparing a third gene expression profile of the treated stem cell population;
comparing the first, second and third gene expression profiles; and
identifying an agent that modulates the expression of a least one gene that is associated with stem cell proliferation.

3. A composition comprising a grouping of nucleic acid molecules that correspond to at least part of the sequences of Table 2 or genes of Table 3 affixed to a solid support.
